(19) United States
(12) Patent Application Publication
(10) Pub. No.: US 2002/0102737 A1
(43) Pub. Date: Aug. 1, 2002

(54) Diagnostic methods for pompe disease and other glycogen storage diseases

(76) Inventors: David S. Millington, Chapel Hill, NC (US); Yan An, Chapel Hill, NC (US); Yuan Tsong Chen, Chapel Hill, NC (US); Robert D. Stevens, Durham, NC (US); Sarah P. Young, Durham, NC (US); Johan L. K. Van Hove, Mol (BE)

Correspondence Address: MYERS BIGEL SIBLEY & SAJOVEC, PO BOX 37428, RALEIGH, NC, 27627, US

(21) Appl. No.: 09/875,327

(22) Filed: Jun. 6, 2001

Related U.S. Application Data
(60) Provisional application No. 60/209,920, filed on Jun. 7, 2000.

(51) Int. Cl. G01N 33/487

(52) U.S. Cl. 436/94; 436/173

(57) ABSTRACT

Provided are methods of screening subjects for lysosomal storage diseases, preferably glycogen storage diseases, using a tetrasaccharide as a biomarker. In a more preferred embodiment, subjects are screened for Pompe disease (i.e., glycogen storage disease type II). Also provided are neonatal screening assays. The present invention further provides methods of monitoring the clinical condition and efficacy of therapeutic treatment in affected subjects. Further provided are methods of measuring a tetrasaccharide biomarker by tandem mass spectrometry, preferably, as part of a neonatal screening assay for Pompe disease.

### RELATED APPLICATION INFORMATION

This application claims the benefit of U.S. Provisional Application No. 60/209,920, filed Jun. 7, 2000, which is incorporated by reference herein in its entirety.

### FIELD OF THE INVENTION

The present invention relates to methods of diagnosing and monitoring subjects with lysosomal storage diseases, in particular, the present invention relates to methods of diagnosing and monitoring subjects with Pompe disease (i.e., glycogen storage disease type II) and other glycogen storage diseases based on the presence of a biomarker in body fluids or tissues.

### BACKGROUND OF THE INVENTION

Pompe disease, also known as glycogen storage disease type II (GSD-II) or acid maltase deficiency, is an inherited disorder of glycogen metabolism resulting from defects in the activity of lysosomal acid α-glucosidase (GAA), a glycogen degrading enzyme (Hirschhorn, R. (1995) in The Metabolic and Molecular Bases of Inherited Disease, 7^{th} Edition, Volume 2 (Scriver, C. R., Beaudet, A. L., Sly, W. S., and Valle, D. Eds), pp. 2443-2464, McGraw-Hill, New York). In its most severe form, the disease is characterized by massive cardiomegaly, macroglossia, progressive muscle weakness and marked hypotonia in early infancy. Most infantile patients are diagnosed between 3-6 months of age and die before 1 year of age.

Recently, a recombinant human precursor, rhGAA produced in Chinese hamster ovary (CHO) cell cultures (Van Hove J L K, et al. (1996) Proc. Natl. Acad. Sci., USA. 93:65-70), and in transgenic mouse and rabbit milk (Bijvoet A G A, et al. (1998) Hum Mol Genet. 7:1815-24; Bijvoet A G A, et al. (1999) Hum Mol Genet. 8:2145-53) has been produced. The rhGAA has been shown to correct the defect in animal models and in patient cells (Kikuchi T, et al. (1998) J Clin Invest 101, 827-833; Bijvoet A G A, et al. (1999) Hum Mol Genet; 8, 2145-53) and a gene therapy vector has been applied to correct all affected muscles in a mouse model (Amalfitano, A. et al. (1999) Proc. Natl. Acad. Sci. U.S.A. 96, 8861-8866). Preliminary study of human Pompe disease patients has demonstrated that rhGAA is capable of improving cardiac and skeletal muscle functions in these patients (Amalfitano A, et al. (2001) Genet Med. 3:132). The promising new treatment has prompted the need for a biomarker assay, suitable for both early diagnosis and treatment monitoring.

At the present time, there is no readily available (and non-invasive) biomarker that may be used in the diagnosis of Pompe disease. The development of a screening assay for Pompe disease would be particularly beneficial in infantile forms of the disease. Early prognosis and treatment of neonates or infants with Pompe disease may improve the prognosis for these patients. Moreover, a method of monitoring therapy may improve the efficacy of treatment and the prognosis for Pompe disease patients.

Using chromatographic methods, Hallgren et al. ((1974) Eur. J. Clin. Invest 4, 429-433), identified and characterized a glucose tetramer, having the presumptive structure: Glcα1-6Glcα1-4Glcα1-4Glc (Glc₄), that was elevated in the urine of a 10-year-old patient with Pompe Disease.

Urinary (Glc)₄ has also been shown to be elevated in glycogen storage diseases type III and type VI (Lennartson, G., et al. (1976) Biomed. Mass Spectrom. 3, 51-54; Oberholzer, K. and Sewell, A. C. (1990) Clin. Chem. 36, 1381), Duchenne muscular dystrophy (Lennartson, G., et al. (1976) Biomed. Mass Spectrom. 3, 51-54; Kikuchi T, et al. (1998) J Clin Invest 101, 827-833), acute pancreatitis (Kumlien et al., (1988) Clin. Chim. Acta 176:39; Kumlien et al., (1989) Int. J. Pancreatol. 4:139; Wang, W. T., et al. (1989) Anal. Biochiem. 182, 48-53), certain malignancies (Kumlien et al., (1988) Clin. Chim. Acta 176:39), and during pregnancy (Zopf, D. A., et al. (1982) J. Immunol. Methods 48, 109-119; Hallgren, P., et al. (1977) J. Biol. Chem. 252, 1034-1040).

Lennartson et al., (1978) Eur. J. Biochem. 83:325 characterized urinary oligosaccharides excreted by two children with GSD type II or type III by gas chromatography (GC)/mass spectroscopy (MS). The primary oligosaccharide secreted in both conditions was (Glc)₄. Larger oligosaccharides were also present. Likewise, Chester et al., (1983) Lancet 1:994 describes a 4-60 fold elevation in urinary (Glc)₄ excretion in patients with GSD type II and type III. These investigators also reported that urinary (Glc)₄ was moderately elevated in clinically normal heterozygotes. Oligosaccharide identification and quantitation was carried out by radioimmunoassay and gas chromatography/mass spectrometry. See also, Peelen et al., (1994) Clin. Chem. 40:914, and Klein et al., (1998) Clin. Chemistry 44:2422.

Oberholzer et al., (1990) Clin. Chem. 36:1381 analyzed urinary (Glc)₄ excretion in patients with GSD using high performance liquid chromatography (HPLC). This report found that (Glc)₄ excretion in urine correlated with hepatic, but not purely muscular, symptoms in patients with GSD.

None of the foregoing studies have evaluated plasma concentrations of (Glc)₄ in GSD patients. Further, these studies do not address whether (Glc)₄ concentrations are elevated as compared with healthy subjects during the neonatal period. Moreover, these references do not suggest that (Glc)₄ may be employed as a biomarker to diagnose Pompe disease, to assess the severity of the disease, or to monitor the clinical condition of a Pompe disease patient, e.g., to assess the effectiveness of a therapeutic regime.

Various methods have been developed to assay (Glc)₄, including gas chromatography-mass spectrometric analysis following permethylation of fractionated urinary oligosaccharides (Lennartson, G., et al. (1976) Biomed. Mass Spectrom. 3, 51-54.), radioimmunoassay (Zopf, D. A., et al. (1982) J. Immunol. Methods 48, 109-119), enzyme-linked immunosorbent assay (Kumlien, J. et al. (1986) Glycoconjugate J. 3, 85-94), HPLC using a monoclonal antibody to (Glc)₄ (Wang, W. T., et al. (1989) Anal. Biochem. 182, 48-53) and HPLC methods involving analysis of perbenzoylated oligosaccharides (Oberholzer, K. and Sewell, A. C. (1990) Clin. Chem. 36, 1381), or employing anion-exchange with pulsed amperometric detection or post column derivatization (Peelen, G. O. H., et al. (1994) Clin. Chem. 40, 914-921). As far as the present inventors are aware, the detection and quantification of (Glc)₄ using tandem mass spectrometry has not previously been described. Moreover, plasma concentrations of (Glc)₄ in Pompe disease patients have not previously been reported. Further, a protocol for using (Glc)₄ as a biomarker for Pompe disease during the neonatal period has not previously been suggested.

Meikle et al., (1997) Clin. Chem. 43:1325 and WO 97/44668 describe the use of a lysosomal membrane protein, LAMP-1, as a general diagnostic marker for lysosomal storage disorders. LAMP-1 concentrations were measured in plasma samples using a time-resolved fluorescence immunoassay in healthy subjects as well as subjects affected with one of twenty-five lysosomal storage disorders. LAMP-1 was elevated in plasma samples in subjects affected with seventeen of the twenty-five disorders evaluated. However, only one of four subjects with Pompe disease that were screened showed an elevation in plasma LAMP-1 concentrations, although all four subjects presented with severe clinical symptoms. LAMP-1 and lysosomal enzyme activities were also characterized in a fibroblast cell line established from a patient with Pompe disease.

Hua et al., (1998) Clin. Chemistry 44:2094 used a second lysosomal membrane protein, LAMP-2, as a biomarker to screen for lysosomal storage disorders. LAMP-2 was measured in plasma from healthy and affected individuals using fluorescence-immunoquantification. Subjects affected with fourteen of twenty-five lysosomal storage disorders evaluated showed an elevation in plasma LAMP-2 concentrations. None of the four subjects with Pompe disease, however, exhibited an elevation in LAMP-2. LAMP-1 and LAMP-2 concentrations were also measured in neonatal blood spots from an “unpartitioned” newborn population. LAMP-1 and LAMP-2 concentrations were elevated in neonates as compared with levels in older subjects. This report suggests that a primary screen with these lysosomal membrane biomarkers may give rise to a high rate of false positives. These investigators suggest that the top 1-5% of the neonatal population be examined further with second-tier diagnostic methods.

Accordingly, there is a need in the art for methods of identifying subjects with Pompe disease, in particular, during the neonatal period. There is also a need in the art for non-invasive methods of identifying and monitoring individuals with Pompe disease. There is further a need in the art for neonatal screening methods for Pompe disease that are compatible with existing methodologies for screening other inherited metabolic disorders.

### SUMMARY OF THE INVENTION

As described in more detail below, the present invention provides a method of screening and monitoring disorders that are characterized by accumulation (i.e., elevated concentrations) of a hexose tetramer biomarker, designated (Glc)₄, in biological samples collected from affected individuals. The (Glc)₄ tetramer is particularly useful as a biomarker for screening and monitoring glycogen storage diseases, e.g., GSD-II (Pompe disease). In preferred embodiments, the inventive methods can be employed for neonatal screening by analysis of (Glc)₄ concentrations in dried blood spots (e.g., on neonatal screening cards).

The presumptive structure of the hexose tetrasaccharide (Glc₄) has been determined as: α-D-Glc(1→6)-α-D-Glc(1→4)-α-D-Glc(1→4)-D-Glc.

The present invention provides the capability to diagnose, detect, and/or monitor Pompe disease in an objective fashion, using fast and reliable methods (e.g., HPLC or tandem mass spectrometry (TMS)), to assay for elevated levels of the (Glc)₄ biomarker. The present invention is advantageous because of its sensitivity, reproducibility, high resolution, simplicity, and low cost over previously-described methods. Moreover, the neonatal screening assays disclosed herein are compatible with current neonatal screening methodologies for other inherited metabolic disorders.

Accordingly, as a first aspect, the present invention provides a method of screening a subject for a glycogen storage disease, comprising the steps of: determining the concentration of hexose tetrasaccharide (Glc)₄ in a biological sample taken from the subject, and comparing the concentration to a reference value, wherein the detection of (Glc)₄ in the biological sample at more than the reference value identifies the subject as affected with a glycogen storage disease. Preferably, the (Glc)₄ tetrasaccharide has the presumptive structure α-D-Glc(1→6)-α-D-Glc(1→4)-α-D-Glc(1→4)-D-Glc. It is further preferred that the glycogen storage disease is glycogen storage disease type II (GSD-II or Pompe disease), GSD III, or GSD VI; more preferably GSD-II.

As a further aspect, the invention provide a method of screening a neonatal subject for Pompe disease (glycogen storage disease type II), comprising the steps of determining the concentration of hexose tetrasaccharide (Glc)₄ in a biological sample taken from the neonatal subject, and comparing the concentration to a reference value; wherein the detection of (Glc)₄ in the biological sample at more than the reference value identifies the neonatal subject as affected with Pompe Disease. Preferably, (Glc)₄ has the presumptive structure α-D-Glc(1→6)-α-D-Glc(1→4)-α-D-Glc(1→4)-D-Glc. It is further preferred that the biological sample is a blood, serum, plasma or urine sample (more preferably, a dried blood, serum, plasma or urine sample).

As a further aspect, the present invention provides a method of monitoring the clinical condition of a subject with Pompe disease (glycogen storage disease II), comprising the steps of: determining the concentration of hexose tetrasaccharide (Glc)₄ in a biological sample taken from the subject, and comparing the concentration to a reference value; wherein the detection of (Glc)₄ in the biological sample at more than the reference value is indicative of the clinical condition of the subject. Preferably, the (Glc)₄ biomarker has the presumptive structure α-D-Glc(1→6)-α-D-Glc(1→4)-α-D-Glc(1→4)-D-Glc. In particular embodiments, this method is practiced to assess the efficacy of a therapeutic regime in the subject.

As a still further aspect, the present invention provides a method of screening a neonatal subject for Pompe disease (glycogen storage disease type II), comprising the steps of: determining the concentration of hexose tetrasaccharide (Glc)₄ by tandem mass spectrometry in a dried blood spot from the neonatal subject, and comparing the concentration to a reference value; wherein the detection of (Glc)₄ in the biological sample at more than the reference value identifies the neonatal subject as affected with Pompe Disease. Preferably, the (Glc)₄ biomarker has the presumptive structure α-D-Glc(1→6)-α-D-Glc(1→4)-α-D-Glc(1→4)-D-Glc.

The (Glc)₄ tetrasaccharide may be quantified or determined by any method known in the art, e.g., tandem mass spectrometry, mass spectrometry, HPLC, immunopurification methods, liquid chromatography, and the like. HPLC and tandem mass spectrometry are preferred, with tandem mass spectrometry being most preferred.

A further aspect of the invention is a method of quantifying or determining the concentration of an oligosaccharide in a biological sample, comprising the step of quantifying or determining the concentration of hexose tetrasaccharide (Glc)₄ by tandem mass spectrometry in a biological sample taken from a subject. Preferably, (Glc)₄ has the presumptive structure α-D-Glc(1→6)-α-D-Glc(1→4)-α-D-Glc(1→4)-D-Glc. It is also preferred that a [U-¹³C]glucose labeled hexose tetramer is used as an internal standard for the TMS protocol.

The methods of the present invention may also be carried out using other oligosaccharides (e.g., limit dextrins) that accumulate in patients with GSD-II as a biomarker.

These and other aspects of the invention are set forth in more detail in the description of the invention below.

### BRIEF DESCRIPTION OF THE DRAWINGS

FIG. 1 shows a chromatogram of HPLC separation of BAB-labeled (Glc)₄ in urine samples. The Y-axis is UV absorbance at 304 nm. The X-axis is time of elution in minutes. I.S. is internal standard of cellopentose (C5) at 10 mg/mL. Panel A is elution profile of urine from a normal individual. Panel B is elution profile of urine from a GSD II patient.

FIG. 2 shows a chromatogram of HPLC separation of BAB-labeled (Glc)₄ in plasma samples. The Y-axis is UV absorbance at 304 nm. The X-axis is time of elution in minutes. I.S. is internal standard of cellopentose (C5) at 1 mg/mL. Panel A is elution profile of urine from a normal individual. Panel B is elution profile of urine from a GSD II patient.

FIG. 3 shows a chromatogram of HPLC analysis of PMP-labeled oligosaccharides, Maltohexose (M₆), Maltopentose (M₅), Maltotetraose (M₄), (Glc)₄, Maltotriose (M₃), Maltose (Mlt), and Glucose (Glc). The Y-axis is UV absorbance at 304 nm. The X-axis is time of elution in minutes. Panel A is elution profile of (Glc)₄ and Malto-oligosaccharide standards. Panel B is elution profile of (Glc)₄ in urine of a GSD II patient. The arrow indicates the absence of M₄.

FIG. 4 shows a product ion spectra of BAB-labeled maltotetraose sodium adduction (M₄-BAB) Na⁺, m/z 866.4 (Panel A); BAB-labeled (Glc)₄ sodium adduction (Glc₄-BAB) Na⁺, m/z 866.4 (Panel B); and hexose tetramer present in GSD II patient urine sample, m/z 866.4 (Panel C). Productions m/z 704.4, m/z 542.3, and m/z 509.2 correspond to losses of one hexose, two hexoses, and BAB-labeled glucose, respectively. The Y-axis is % Intensity of fragments. The X-axis is m/z values.

FIG. 5 shows an ESI-MS-MS spectra of BAB-labeled oligosaccharides in the urine of a glycogen storage disease type II patient. The derivative sample was directly injected into ESI-MS-MS after C18 cartridge purification. The ions were scanned by a quadrupole mass spectrometer (see text for experimental details). The Y-axis is % Intensity of fragments. The X-axis is m/z values.

FIG. 6 shows the (Glc)₄ levels in urine from patient 1 (Panel A), patient 2 (Panel, B), and patient 3 (Panel C). (Glc)₄ levels are in mmol/mol creatinine (Cr). Dashed line represents the main (Glc)₄ levels plus standard deviation in 20 normal controls (<1 year old). Open arrow indicates the start of enzyme therapy treatment. Closed arrow with dashed line indicates the start of double enzyme doses. Closed arrow with solid line indicates the start of immunotherapy.

FIG. 7 shows the (Glc)₄ levels in plasma from patient 1 (Panel A), patient 2 (Panel B), and patient 3 (Panel C). (Glc)₄ levels are in mg/mL. Dashed line represents the main (Glc)₄ levels plus standard deviation in 20 normal controls (<1 year old). Open arrow indicates the start of enzyme therapy treatment. Closed arrow with dashed line indicates the start of double enzyme doses. Closed arrow with solid line indicates the start of immunotherapy.

FIG. 8 is a graphical representation of BAB-derivatives of the tetrasaccharide fraction of the internal standard reaction mixture separated by HPLC.

FIG. 9 shows a comparison of Glc₄ analysis in control and patient urine samples by either HPLC or ESI-MS/MS.

FIG. 10 shows a comparison of Glc₄ analysis in control and patient plasma by either HPLC or ESI-MS/MS.

FIG. 11 shows a comparison of Glc₄ analysis in paired liquid and spotted urine samples by ESI-MS/MS.

FIG. 12 shows the putative structure of the Glc₄ tetrasaccharide.

### DETAILED DESCRIPTION OF THE INVENTION

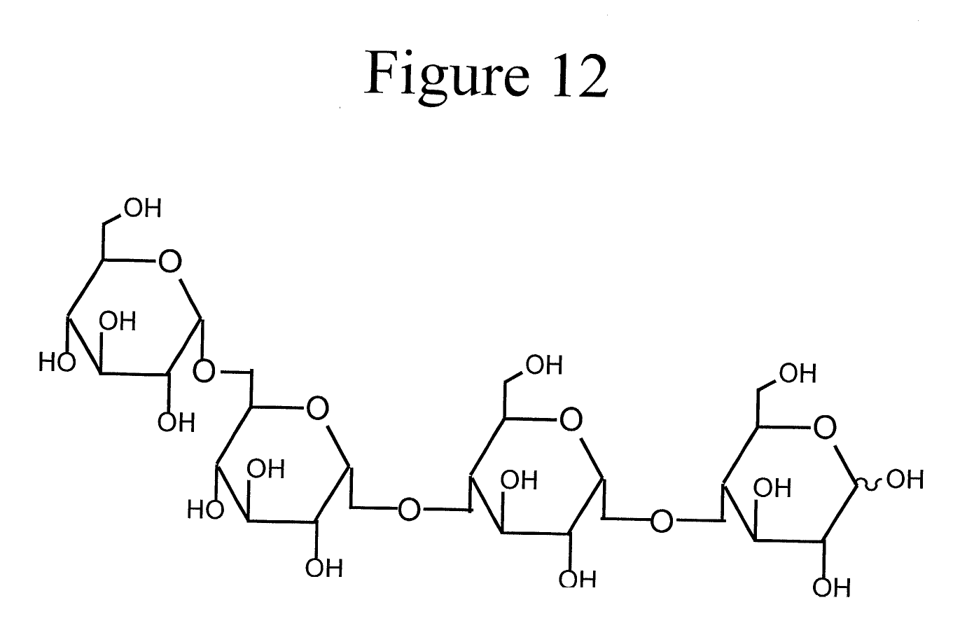

The present invention is based, in part, on the discovery that a hexose tetramer {hereinafter, (Glc)₄} may be used as a biomarker for screening methods of detecting glycogen storage disease type II (GSD-II). (Glc)₄ has been presumptively identified as a glucose tetrasaccharide. The evidence further indicates that (Glc)₄ oligosaccharide has the structure α-D-Glc(1→6)-(α-D-Glc(1→4)-α-D-Glc(1→4)-D-Glc (Hallgren et al. (1974) Eur. J. Clin. lnvest. 4:429; see FIG. 12).

The present investigations have found that (Glc)₄ concentrations, in particular plasma (Glc)₄ concentrations, may be used to monitor Pompe disease patients (e.g., to assess the efficacy of a therapeutic regime); (Glc)₄ concentrations may be well-correlated with the clinical course of the disease in affected patients.

The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise.

The terms “Pompe disease” and “glycogen storage disease type II” (i.e., GSD-II) are used interchangeably herein, although “Pompe disease” is conventionally used more frequently to designate the infantile form of the disorder.

The term (Glc)₄, as used herein, refers to a hexose tetramer {(hex)₄} biomarker that accumulates in biological fluids (e.g., urine and plasma) of Pompe disease patients. (Glc)₄ has been presumptively identified as a glucose tetrasaccharide (e.g., a limit dextrin) that accumulates as the result of incomplete glycogen degradation, due to deficiency of the GAA enzyme. The presumptive structure of (Glc)₄ is α-D-Glc(1→6)-α-D-Glc(1→4)-α-D-Glc(1→4)-D-Glc (FIG. 12).

Those skilled in the art will appreciate that the presumptive structure of (Glc)₄, as determined by tandem mass spectrometry of its butyl-p-aminobenzoate derivative, is that of a hexose tetramer. The identify of the hexose constituents and the linkages therebetween cannot be determined by the TMS analysis. Thus, those skilled in the art will appreciate that (Glc)₄ may include any combination of hexose monomers (e.g., glucose, galactose, mannose) linked by any of the possible glycosidic bonds between such monomers (e.g., 1→2, 1→3, 1→4, 1→6).

The association between (Glc)₄ and Pompe Disease, as well as previous observations reported in the literature, strongly suggest that a glucose tetrasaccharide is a significant component of (Glc)₄. “Screening” as used herein refers to a procedure used to evaluate a subject for the presence of a disorder characterized by accumulation of (Glc)₄, as described above. It is not required that the screening procedure be free of false positives or false negatives, as long as the screening procedure is useful and beneficial in determining which of those individuals within a group or population of individuals are affected with a particular disorder. The screening methods disclosed herein may be diagnostic and/or prognostic methods and/or may be used to monitor patient therapy.

A “diagnostic method”, as used herein, refers to a screening procedure that is carried out to identify those subjects that are affected with a particular disorder.

A “prognostic method” refers to a method used to help predict, at least in part, the course of a disease. Alternatively stated, a prognostic method may be used to assess the severity of the disease. For example, the screening procedure disclosed herein may be carried out to both identify an affected individual, to evaluate the severity of the disease, and/or to predict the future course of the disease. Such methods may be useful in evaluating the necessity for therapeutic treatment, what type of treatment to implement, and the like. In addition, a prognostic method may be carried out on a subject previously diagnosed with a particular disorder when it is desired to gain greater insight into how the disease will progress for that particular subject (e.g., the likelihood that a particular patient will respond favorably to a particular drug treatment, or when it is desired to classify or separate patients into distinct and different sub-populations for the purpose of conducting a clinical trial thereon).

The terms “quantifying the concentration” or “determining the concentration,” as used herein, refer to measurement of the concentration or level of the analyte in the indicated sample. Typically, an absolute or relative numerical value will be assigned to the concentration of the analyte in the sample as a result of the quantifying or determining step. Any suitable method known in the art may be used to quantify or determine the concentration of (Glc)₄ in a biological sample according to the present invention, as described in more detail hereinbelow. Methods of “quantifying” or “determining” the concentration of (Glc)₄ encompass both quantitative and or semi-quantitative methodologies, also as described in more detail below.

A “quantitative” method is one that assigns an absolute or relative numerical value to the concentration of the analyte in the biological sample.

A “semi-quantitative” method is one that indicates that the concentration of the analyte is above a threshold level, but does not assign an absolute or relative numerical value. Analytical methods that are commonly known as “dipstick” methods are examples of semi-quantitative assays.

The following description of the invention is directed to the (Glc)₄ oligosaccharide. The methods of the invention may also be applied to the use of longer oligosaccharides (i.e., any limit dextrin produced by incomplete glycogen degradation due to a deficiency of the GAA enzyme) for the detection of Pompe disease.

For example, (Glc)₆, (Glc)₇ and (Glc)₈ have been described in the urine of patients with Pompe disease (Lennartson et al., (1978) Eur. J. Biochem. 83:325; Kumlien et al., (1989) Arch. Biochem. Biophys. 269:678). At least three (Glc)₆ isomers exist having the presumptive structures: (α-D-Glc(1→6)-α-D-Glc(1→6)-α-D-Glc(1→4)-α-D-Glc(1→4)-α-D-Glc(1→4)-D-Glc, α-D-Glc(1→4)-α-D-Glc(1→6)-α-D-Glc(1→6)-α-D-Glc(1→4)-α-D-Glc(1→4)-D-Glc, and α-D-Glc(1→6)-α-D-Glc(1→4)-α-D-Glc(1→6)-α-D-Glc(1→4)-α-D-Glc(1→4)-D-Glc. The presumptive structure of (Glc)₇ isomers have been determined as α-D-Glc(1→6)-α-D-Glc(1→4)-α-D-Glc(1→4)(α-D-Glc(1→6))-α-D-Glc(1→4)-D-Glc-α-D-Glc(1→4)-D-Glc and α-D-Glc(1→6)-α-D-Glc(1→4)-α-D-Glc(1→4)-α-D-Glc(1→6)-α-D-Glc(1→4)-D-Glc-α-D-Glc(1→4)-D-Glc. The presumptive structure of the (Glc)₈ oligosaccharide has been determined to be: α-D-Glc(1→6)-α-D-Glc(1→4)-α-D-Glc(1→4)-α-D-Glc(1→4)-α-D-Glc(1→6)-α-D-Glc(1→4)-α-D-Glc(1→4)-D-Glc. A (hex)₅ oligosaccharide (or oligosaccharides) of unknown structure(s) has been detected in the urine and plasma of Pompe disease patients and controls by TMS and is likely to be a limit dextrin of glycogen. Hexose oligomers with up to 11 residues have been detected in the urine of a Pompe disease patient by matrix assisted laser desorption—time of flight mass spectrometry (Klein et al., (1998) Clin. Chem. 44:2422).

Those skilled in the art will appreciate that oligosaccharides with an α-(1→6) glycosidic bond at the non-reducing end are more stable and preferred. Likewise, longer oligosaccharides will tend to be less stable than shorter oligosaccharides. The degree to which the particular oligosaccharide accumulates in biological samples from healthy as compared with Pompe disease patients is a further consideration. Finally, the existence of interfering substances may further inform the choice of oligosaccharide for use as a biomarker in accordance with the present invention.

In general, as an alternative to (Glc)₄, the (Glc)₅, (Glc)₆, (Glc)₇, (Glc)₈ and longer chain hexose oligosaccharides are the preferred biomarkers, in particular, with neonatal subjects. Alternatively, these longer chain oligosaccharides may be utilized as a secondary biomarker, e.g., to identify false positives using the (Glc)₄ assay. These longer hexose oligomers may be measured using similar protocols for the detection of (Glc)₄. For example, with respect to tandem mass spectrometry, the same derivitization and scan functions may be employed, but with different masses (m/z) detected.

(Glc)₄: A Biomarker for Pompe Disease and Other Metabolic Disorders

The (Glc)₄ tetrasaccharide is believed to have a glycogen origin and to represent a by-product of incomplete glycogen degradation (i.e., a limit dextrin) as a result of acid lysosomal α-glucosidase (GAA) deficiency in Pompe disease patients. Evidence suggests this limit dextrin is formed when glycogen is released into the circulation, possibly as a result of cell lysis caused by the accumulation of glycogen in the lysosomes. In the circulation glycogen is acted upon by α-amylase and neutral α1-4 glucosidase resulting in the production of limit dextrins (Ugorski, (1983) J. Exp. Pathol. 1:27). (Glc)₄ may be found at elevated concentrations in body fluids (e.g., blood, plasma, serum, urine, sputum, amniotic fluid, and the like).

Accumulation of the (Glc)₄ tetrasaccharide in urine has also been associated with other glycogen storage diseases and disorders, e.g., GSD-III and GSD-VI, Duchenne muscular dystrophy, acute pancreatitis, and in certain malignancies. GSD-III is caused by a deficiency in glycogen debranching enzyme activity. GSD-VI is a heterogeneous group of diseases caused by a deficiency of the liver phosphorylase system. The deficiency may be in the liver phosphorylase enzyme itself or in phosphorylase kinase.

Accordingly, the present invention provides a method of screening a subject for a disorder that is characterized by an accumulation (i.e., elevated concentration) of (Glc)₄ in a biological sample collected from the subject. According to this method, a biological sample is collected from a subject, and the presence or absence of (Glc)₄ in the sample is determined, where the presence of (Glc)₄ in the sample presumptively identifies the subject as affected with the disorder.

Alternatively, and preferably, the method may be quantitative or semi-quantitative in nature. According to this embodiment, a biological sample is obtained from a subject, and the concentration of (Glc)₄ in the biological sample is quantified or determined. Levels of (Glc)₄ in the biological sample over a reference value (i.e., reference concentration) presumptively identifies the subject as affected with the disorder. Typically, the reference value will be based on known concentrations of (Glc)₄ in healthy and affected populations, as appropriate for the subject being screened (e.g., a neonatal subject will, in general, be compared with a healthy and/or affected neonatal population). For example, the subject may be compared with a matched, unselected, population. Alternatively, the subject may be compared with a matched population of unaffected (i.e., healthy) subjects and/or a matched population of affected subjects.

It is preferred that subjects are compared with an age-matched population as there is a trend towards reduced (Glc)₄ levels with age in healthy subjects (see Tables 4 and 5). Those skilled in the art will also appreciate that (Glc)₄ levels may be higher in patients with early onset of Pompe disease as compared with later onset forms of the disease.

The reference value may be selected according to any method known in the art. In particular embodiments, the reference value may be a predetermined value. Alternatively, the reference value may be determined during the course of the assay. For example, samples from known unaffected and/or affected subjects may be run concurrently with the test samples and a reference value determined therefrom. As a further alternative, test samples from a mixed population may be analyzed, and the reference value is determined based on the distribution of the results, e.g., using statistical methods as known in the art.

Thus, the reference value represents a threshold value for identifying affected subjects. The choice of the reference value is not absolute. For example, a relatively low value may advantageously reduce the incidence of false negatives, but may also increase the likelihood of false positives. Accordingly, as for other screening techniques, the reference value may be based on a number of factors, including but not limited to cost, the benefit of early diagnosis and treatment, the invasiveness of follow-up diagnostic methods for individuals that have false positive results, and other factors that are routinely considered in designing screening assays.

Subjects may be presumptively identified as affected using any method known in the art. For example, subjects that have (Glc)₄ values above about the 70^{th} percentile, 80^{th} percentile, 90^{th} percentile, 95^{th} percentile, 96^{th} percentile, 97^{th} percentile, 98^{th} percentile, 99^{th} percentile, or higher, as compared with an appropriate matched control population may be presumptively identified as affected. Alternatively, subjects having more than about a 2, 3, 4, 5, 8, 10 or 20 fold higher (Glc)₄ concentrations than the average (alternatively, mean or median) value for an appropriate unaffected population may be presumptively identified as affected.

Secondary biomarkers may optionally be used to identify likely false positives in the (Glc)₄ assay. Exemplary secondary biomarkers include the longer chain oligosaccharides (described above) found in body fluids of Pompe disease patients. Other possible secondary biomarkers include the LAMP-1 and LAMP-2 markers (Meikle et al., (1997) Clin. Chem. 43:1325 and WO 97/44668; Hua et al., (1998) Clin. Chemistry 44:2094).

In preferred embodiments, the foregoing methods are carried out to screen subjects for lysosomal storage diseases (e.g., glycogen storage diseases) or Duchenne muscular dystrophy, more preferably, glycogen storage diseases (other than GSD-I), still more preferably GSD-II (Pompe disease), GSD-III or GSD-VI. In the most preferred embodiment, the method is employed to screen subjects for Pompe disease (GSD-II).

There are a multitude of lysosomal storage diseases that are known in the art. Exemplary lysosomal storage disease include, but are not limited to, GM1 gangliosidosis, Tay-Sachs disease, GM2 gangliosidosis (AB variant), Sandhoff disease, Fabry disease, Gaucher disease, metachromatic leukodystrophy, Krabbe disease, Niemann-Pick disease (Types A-D), Farber disease, Wolman disease, Hurler Syndrome (MPS III), Scheie Syndrome (MPS IS), Hurler-Scheie Syndrome (MPS IH/S), Hunter Syndrome (MPS II), Sanfilippo A Syndrome (MPS IIIA), Sanfilippo B Syndrome (MPS IIIB), Sanfilippo C Syndrome (MPS IIIC), Sanfilippo D Syndrome (MPS IIID), Morquio A disease (MPS IVA), Morquio B disease (MPS IV B), Maroteaux-Lamy disease (MPS VI), Sly Syndrome (MPS VII), (α-mannosidosis, β-mannosidosis, fucosidosis, aspartylglucosaminuria, sialidosis (mucolipidosis I), galactosialidosis (Goldberg Syndrome), Schindler disease, mucolipidosis II (I-Cell disease), mucolipidosis III (pseudo-Hurler polydystrophy), cystinosis, Salla disease, infantile sialic acid storage disease, Batten disease (juvenile neuronal ceroid lipofuscinosis), infantile neuronal ceroid lipofuscinosis, mucolipidosis IV, and prosaposin.

Enzyme deficiencies that are associated with lysosomal storage diseases according to the present invention include, but are not limited to, deficiencies in β-galactosidase, β-hexosaminidase A, β-hexosaminidase B, GM₂ activator protein, glucocerebrosidase, arylsulfatase A, galactosylceramidase, acid sphingomyelinase, acid ceramidase, acid lipase, α-L-iduronidase, iduronate sulfatase, heparan N-sulfatase, α-N-acetylglucosaminidase acetyl-CoA, glucosaminide acetyltransferase, N-acetylglucosaminidase-6-sulfatase, arylsulfatase B, β-glucuronidase, α-mannosidase, β-mannosidase, α-L-fucosidase, N-aspartyl-β-glucosaminidase, α-neuraminidase, lysosomal protective protein, α-N-acetyl-galactosaminidase, N-acetylglucosamine-1-phosphotransferase, cystine transport protein, sialic acid transport protein, the CLN3 gene product, palmitoyl-protein thioesterase, saposin A, saposin B, saposin C, or saposin D.

There are numerous glycogen storage diseases known, see e.g., Y. T. Chen & A. Burchell, Glycogen storage diseases. In: C. R. Scriver et al. (Eds.). The Metabolic and Molecular Bases of Inherited Disease, 7^{th} ed. New York: McGraw-Hill. 1995, pp.935-965. Exemplary glycogen storage diseases include, but are not limited to, Type Ia GSD (von Gierke disease), Type Ib GSD, Type Ic GSD, Type Id GSD, Type II GSD (including Pompe disease or infantile Type II GSD), Type IIIa GSD, Type IIIb GSD, Type IV GSD, Type V GSD (McArdle disease), Type VI GSD, Type VII GSD, glycogen synthase deficiency, hepatic glycogenosis with renal Fanconi syndrome, phosphoglucoisomerase deficiency, muscle phosphoglycerate kinase deficiency, phosphoglycerate mutase deficiency, and lactate dehydrogenase deficiency.

Enzyme deficiencies that are associated with glycogen storage diseases include, but are not limited to, deficiencies in glucose 6-phosphatase, lysosomal acid a glucosidase, glycogen debranching enzyme, branching enzyme, muscle phosphorylase, liver phosphorylase, phosphorylase kinase, muscle phosphofructokinase, glycogen synthase phosphoglucoisomerase, muscle phosphoglycerate kinase, phosphoglycerate mutase, or lactate dehydrogenase.

Preferably, the present invention is used to detect subjects that have a lysosomal acid α-glucosidase (GAA) deficiency, the metabolic defect in Pompe disease (i.e., GSD-II).

As a further aspect, the present invention provides a method of screening a subject for Pompe disease, comprising quantifying or determining the concentration of (Glc)₄ in a biological sample obtained from the subject. The concentration of (Glc)₄ in the biological sample collected from the subject is compared with a reference value (as this term is described above). Detection of (Glc)₄ concentrations in the biological sample at more than this reference value (which may be a predetermined value) presumptively identifies the subject as affected with Pompe disease.

In general, the methods disclosed herein have both veterinary and medical applications. Accordingly, subjects may be humans, simians, canines, felines, equines, bovines, ovines, caprines, porcines, lagomorphs, rodents, avians, and the like. Typically, however, subjects according to the present invention will be human subjects, e.g., neonatal (i.e., from the time of birth to about one week post-natal), infant, juvenile, adolescent or adult subjects. Neonatal subjects are preferred. As used herein, “neonatal” subjects include premature infants, as that term is used in the art.

The subjects may be part of a general population, e.g., for a broad-based screening assay. Alternatively, the subject may be one that is suspected of having a metabolic disorder characterized by the accumulation of (Glc)₄ (e.g., the subject has clinical symptoms) as described above (e g., a glycogen storage disease, more particularly, GSD-II). In other particular embodiments, subjects have already been diagnosed as having a disorder characterized by accumulation of (Glc)₄ (e.g., to monitor the clinical condition of the patient or the efficacy of the treatment). According to this embodiment, it is preferred that the subject has been diagnosed with a glycogen storage disorder (more preferably, GSD-II).

As used herein, the “biological sample” may comprise any suitable body fluid, cells, or tissue (including cultured cells and tissues) in which (Glc)₄ accumulation may be detected in the disorders described herein (e.g., glycogen storage disorders such as GSD-II, GSD-III, and GSD-VI). Preferably, the biological sample may be obtained by relatively non-invasive methods (i.e., methods that do not involve surgical methods or biopsy), which are less traumatic to the subject, and more suitable for a broad-based screening assay. It is also preferred that the biological sample is a body fluid sample. Exemplary body fluid samples include but are not limited to plasma, sera, blood (including cord blood), urine, sputum, amniotic fluid, and the like. Blood, plasma, sera, and urine samples are more preferred.

Alternatively, the biological sample is a cell or tissue sample, including cultured cells (e.g., fibroblasts) or tissues, and conditioned medium or effusions collected from cells or tissues. Exemplary cells or tissues include, muscle (e.g., skeletal, smooth, cardiac and diaphragm), liver, skin, foreskin, umbilical cells or tissue, and the like. Liver and muscle cells and tissues are preferred.

As a further alternative, the biological sample may be provided on a solid medium, e.g., a filter paper, swab, cotton, and the like. In particular preferred embodiments, the biological sample is a dried blood sample from a neonatal subject, e.g., dried blood spots on neonatal screening cards (i.e., “Guthrie” cards). As a further preferred example, the biological sample may be a dried urine sample (e.g., on a filter paper or lining from a diaper).

Subjects are presumptively identified as affected with a particular disorder (e.g., Pompe disease) by the inventive screening methods described herein. In particular embodiments, additional, second-tier diagnostic testing will be carried out to confirm the diagnosis in these subjects. Typically, such second-tier methodologies (e.g., enzyme assays on tissue biopsies) are more costly, time-consuming and invasive than the screening methods disclosed herein. For example, subjects having (Glc)₄ levels above a reference concentration may be presumptively identified as affected with Pompe disease, and selected for additional diagnostic testing to confirm this diagnosis, assess whether the subject is affected with another disorder (e.g., GSD-III), or is a healthy subject giving a false positive result in the screening assay.

The present invention further finds use in methods of monitoring the clinical course of a subject that has already been positively diagnosed as affected with a disorder characterized by the accumulation of (Glc)₄, as this term is described above. The present investigations have provided the discovery that elevated (Glc)₄ concentrations in biological samples (in particular, plasma, blood and sera) from affected subjects correlates with the clinical state of the affected subject. Indeed, (Glc)₄ concentrations may be elevated prior to the exacerbation of other symptomology in the affected subject, and may be used as an early indicator of regression. Thus, (Glc)₄ may be used as an index of treatment efficacy and the clinical condition of the patient.

Accordingly, the present invention further encompasses methods of monitoring the clinical status of a subject with a disorder characterized by the accumulation of (Glc)₄. Preferably, the subject has already been diagnosed with a glycogen storage disorder, more preferably, GSD-II. The clinical condition of the subject may be monitored to determine the efficacy of a treatment regime, e.g., enzyme replacement therapy, gene therapy, and/or dietary therapy. For example, if levels of the biomarker suggest that the current therapeutic regime is not effective, it may be determined to initiate an altered course of treatment. Alternatively, the condition of the subject may be monitored to determine whether to commence or re-initiate treatment of the subject.

The inventive screening methods disclosed herein may be carried out using any suitable methodology that detects the presence or absence of (Glc)₄ (preferably, determines the concentration of (Glc)₄) in a biological sample (as described above). Illustrative methods include, but are not limited to, chromatographic methods (e.g., high performance liquid chromatography), immunoassay (e.g., immunoaffinity chromatography, immunoprecipitation, radioimmunoassay, immunofluorescence assay, immunocytochemical assay, immunoblotting, enzyme-linked immunosorbent assay (ELISA) and the like), liquid chromatography-mass spectrometry; gas chromatography-mass spectrometry, time-of-flight mass spectrometry, tandem mass spectrometry, and combinations of these mass spectrometry techniques with immunopurification.

Preferred methods will be simple, rapid, accurate, relatively non-invasive (e.g., non-surgical), sensitive, and preferably minimize interfering signals from molecules other than (Glc)₄. When used as a method of neonatal screening, it is further preferred that the methodology is compatible with existing screening assays and is adaptable to automation and high through-put screening of samples.

The methods may be completely manual, alternatively and preferably, they are partially or completely automated. Screening programs to evaluate a large number of samples (e.g., neonatal screening programs) will generally be at least partially automated to facilitate high throughput of samples. Typically, for example, the data will be captured and analyzed using an automated system. In other preferred high throughput methods, arrays or micro-arrays of spotted biological samples (e.g., blood, plasma, serum, urine and the like) may be analyzed concurrently. Such arrays or microarrays may contain greater than about 10, 50, 100, 200, 300, 500, 800, 1000, 2000, 5000 samples or more.

Methods employing HPLC, time-of-flight mass spectrometry, and tandem mass spectrometry (TMS) are preferred, with TMS being most preferred.

A preferred HPLC method for analysis of (Glc)₄ and other glycans in biological samples employs a C18 reversed-phase column. According to this method, baseline separation of standards from monomers (glucose) to heptamers (maltoheptaose) can be readily achieved using derivatives of para-amino-benzoic acid (PABA) and monitoring at a wavelength of 304 nm with a ultraviolet detector.

Preferred methods of quantifying or determining (Glc)₄ and other glycans in biological samples using TMS are described in more detail hereinbelow.

In biological samples in which the concentration of (Glc)₄ analyte is low relative to the limits of detection of the technique, it is preferred to use a concentration step prior to the step of detecting (alternatively, quantifying) (Glc)₄ in the sample. As an illustrative, and preferred, example of a concentration technique, immunoaffinity methods may be used to increase the (Glc)₄ concentration in the sample prior to the detection/quantification step. For example, immunoprecipitation may be carried out with an antibody that specifically recognizes (Glc)₄ conjugated to magnetized beads. Specific anti-(Glc)₄ antibodies are known in the art (see, e.g., Zopf et al., (1982) J. Immunological Methods 18:109; Lundblad et al., (1984) J. Immunological Methods 68:217; Lundblad et al., (1984) J. Immunological Methods 68:227). Size exclusion chromatography may also be used to concentrate the (Glc)₄ in the sample.

These concentration methods may also be used to separate the (Glc)₄ analyte from contaminants or interfering substances.

A further aspect of the invention are antibodies that specifically recognize and bind to (Glc)₄. The term “antibodies” as used herein refers to all types of immunoglobulins, including IgG, lgM, IgA, IgD, and IgE. Of these, IgM and lgG are particularly preferred. The antibodies may be monoclonal or polyclonal and may be of any species of origin, including (for example) mouse, rat, rabbit, horse, or human, or may be chimeric antibodies. See, e.g., M. Walker et al., Molec. Immunol. 26, 403-11 (1989). The antibodies may be recombinant monoclonal antibodies produced according to the methods disclosed in Reading U.S. Pat. No. 4,474,893, or Cabilly et al., U.S. Pat. No. 4,816,567. The antibodies may also be chemically constructed by specific antibodies made according to the method disclosed in SegAl et al., U.S. Pat. No. 4,676,980.

Antibody fragments which contain specific binding sites for (Glc)₄ may also be generated. For example, such fragments include, but are not limited to, the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed to allow rapid and easy identification of monoclonal Fab fragments with the desired specificity (W. D. Huse et al., Science 254, 1275-1281 (1989)).

For the production of antibodies, various hosts including goats, rabbits, rats, mice, humans, and others, may be immunized by injection with (Glc)₄ or a derivative thereof which has immunogenic properties (e.g., conjugated to a hapten or opsonin). Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, keyhole limpet hemocyanin, and dinitrophenol. Among adjuvants used in humans, BCG (bacilli Calmette-Guerin) and Corynebacterium parvum are especially preferable.

Monoclonal antibodies to (Glc)₄ may be prepared using any technique which provides for the production of antibody molecules by continuous cell lines in culture. These include, but are not limited to, the hybridoma technique, the human B-cell hybridoma technique, and the EBV-hybridoma technique (Kohler, G. et al. (1975) Nature 256:495-497; Kozbor, D. et al. (1985) J. Immunol. Methods 81:31-42; Cote, R. J. et al. (1983) Proc. Natl. Acad. Sci. 80:2026-2030; Cole, S. P. et al. (1984) Mol. Cell Biol. 62:109-120). Briefly, the procedure is as follows: an animal is immunized with (Glc)₄ or an immunogenic derivative or conjugate thereof (e.g., conjugated to a hapten or opsonin). Lymphoid cells (e.g. splenic lymphocytes) are then obtained from the immunized animal and fused with immortalizing cells (e.g. myeloma or heteromyeloma) to produce hybrid cells. The hybrid cells are screened to identify those that produce the desired antibody.

Human hybridomas that secrete human antibody can be produced by the Kohler and Milstein technique. Although human antibodies are especially preferred for treatment of humans, in general, the generation of stable human-human hybridomas for long-term production of human monoclonal antibody can be difficult. Hybridoma production in rodents, especially mouse, is a well established procedure and thus, stable murine hybridomas provide an unlimited source of antibody of select characteristics. As an alternative to human antibodies, the mouse antibodies can be converted to chimeric murine/human antibodies by genetic engineering techniques. See V. T. Oi et al., Bio Techniques 4(4):214-221 (1986); L. K. Sun et al., Hybridoma 5 (1986).

The monoclonal antibodies specific for (Glc)₄ can be used to produce anti-idiotypic (paratope-specific) antibodies. See e.g., McNamara et al., Science 220, 1325-26 (1984), R. C. Kennedy, et al., Science 232, 220 (1986).

In addition, techniques developed for the production of “chimeric antibodies”, the splicing of mouse antibody genes to human antibody genes to obtain a molecule with appropriate antigen specificity and biological activity can be used (S. L. Morrison, et al. Proc. Natl. Acad. Sci. 81, 6851-6855 (1984); M. S. Neuberger et al., Nature 312:604-608 (1984); S. Takeda, S. et al., Nature 314:452-454 (1985)). Alternatively, techniques described for the production of single chain antibodies may be adapted, using methods known in the art, to produce (Glc)₄-specific single chain antibodies. Antibodies with related specificity, but of distinct idiotypic composition, may be generated by chain shuffling from random combinatorial immunoglobin libraries (D. R. Burton, Proc. Natl. Acad. Sci. 88,11120-3 (1991)).

Antibodies may also be produced by inducing in vivo production in the lymphocyte population or by screening immunoglobulin libraries or panels of highly specific binding reagents as disclosed in the literature (R. Orlandi et al., Proc. Natl. Acad. Sci. 86, 3833-3837 (1989)); G. Winter et al., Nature 293-299 (1991)).

Neonatal Screening

The methods disclosed herein may be advantageously employed as part of a neonatal screening program to identify affected individuals in the neonatal period so as to permit early medical intervention. Many neonatal screening programs relay on a unique method of specimen collection, in which blood from a heel prick is absorbed onto a neonatal screening card (e.g., a cotton-fiber filter paper). The avoidance of mortality and morbidity caused by defects of amino acids metabolism, such as phenylalanine hydroxylase deficiency, which causes phenylketonuria (PKU), and branched-chain ketoacid dehydrogenase deficiency, which causes maple syrup urine disease (MSUD) is owed to the development of simple biochemical tests for elevated amino acid levels in these dried neonatal blood spots.

The screening methods disclosed herein may further advantageously be performed concurrently or in parallel (i.e., from the same sample but not necessarily in the same assay) with other neonatal screening assays, eg., on neonatal blood samples or dried blood spots on neonatal screening cards.

The neonatal screening card may be of any suitable natural material or synthetic material, including but not limited to cotton, cellulose, acetate, and combinations thereof.

Alternatively, a neonatal screening program may be based on measuring (Glc)₄ in any other biological sample, as described above. For example, (Glc)₄ may be measured in blood (e.g., cord blood), plasma, serum, or urine. In particular embodiments, the urine may be extracted from diaper material.

Accordingly, a further aspect of the invention is a method of screening a neonatal subject for a disorder characterized by the accumulation of (Glc)₄ (as described above), comprising the step of quantifying or determining the concentration of (Glc)₄ in a biological sample taken from the neonatal subject, wherein the detection of (Glc)₄ in the biological sample at more than a reference concentration identifies the neonatal subject as affected with the disorder.

A preferred method of screening a neonatal subject for Pompe disease comprises the step of quantifying or determining the concentration of (Glc)₄ in a blood sample taken from the neonatal subject, wherein the detection of (Glc)₄ in the biological sample at more than a reference value identifies the neonatal subject as affected with Pompe disease. Preferably, the blood sample is taken from a neonatal screening card. As described above, this method may identify neonatal subjects with other disorders, such as GSD-III and other glycogen storage diseases. This feature of the inventive methods does not detract from application of these screening methodologies and, indeed, may be considered a beneficial advantage. Alternatively, other biological samples from a neonatal subject may be employed, e.g., urine (e.g., collected on a piece of diaper material).

In still a further preferred embodiment, described in more detail below, methodologies involving tandem mass spectrometry are utilized as part of a neonatal screening program for GSD-II (and/or other glycogen storage diseases) using (Glc)₄ as a biomarker for the presence of the disease.

As described above, it is preferred that methods of neonatal screening be at least partially automated (as described above). For example, once a sample is loaded onto an HPLC column or into the tandem mass spectrometer, it is preferred that the data be captured and analyzed using an automated system.

Methodologies Based on Tandem Mass Spectrometry (TMS)

TMS is a preferred methodology for carrying out the inventive methods described hereinabove. The concept of TMS for analysis of mixtures using triple quadrupole mass spectrometers was originated by Yost and Enke, Tandem quadrupole mass spectrometry. In: Tandem Mass Spectrometry, F. W. McLafferty (Ed.), Wiley & Sons, New York, (1983), pp. 175-195. For the selective detection of compounds of a similar structural type, either a precursor ion scan function to identify the molecular species that fragment to a common product ion, or a constant neutral loss scan function to identify ions that lose a common fragment, or a multiple reaction monitoring where selected precursor and product ions only are detected is employed. Addition of appropriate internal standards, such as stable isotope-labeled analogs, to the biological matrix before work-up and analysis facilitates accurate quantification of the target analytes.

Any suitable TMS methodology known in the art may be employed, including, but not limited to triple quadrupole mass spectrometry and hybrid mass spectrometry methods that combine quadrupole and time-of-flight mass spectrometers. Ion traps and ion cyclotron resonance mass spectrometers can also be employed.

TMS is particularly suitable to neonatal screening programs. The ability to quantify amino acids and acylcarnitines alone enables more than twenty metabolic disorders to be recognized. In a collaborative retrospective study, it has been confirmed that PKU (Chace et al., (1993) Clin. Chem. 39:66), MSUD (Chace et al., (1995) Clin. Chem. 41:62), hypermethioninemias (Chace et al., (1996) Clin. Chem. 43:2106), and medium-chain acyl-coA dehydrogenase deficiency (MCAD) (Chace et al., (1997) Clin. Chem. 43:2106) can all be reliably detected by TMS in the neonatal period (see also, Sweetman, (1996) Clin. Chem. 42:345). The analytes are simultaneously quantified by TMS using interlaced scan function as the sample mixture is injected into a flowing solvent. A batch process has been reported that prepares and analyzes samples in a 96-well format, uses an automated computer algorithm to interpret results, and has demonstrated the ability to analyze up to 1000 samples per day (Rashed et al., (1997) Clin. Chem. 43:1129). Neonatal screening using TMS has been implemented in a variety of jurisdictions.

As far as the present inventors are aware, the HPLC and TMS studies described herein are the first to report elevated (Glc)₄ concentrations in plasma (or other blood-derived) samples from subjects affected with Pompe disease. Further disclosed herein is the first TMS protocol for screening for Pompe disease using (Glc)₄ as a biomarker, in particular, the first such neonatal screening program.

Accordingly, the present invention further provides a method of quantifying or determining the concentration of (Glc)₄ in a biological sample by TMS. The oligosaccharides in the sample may be derivatized prior to analysis by any method known in the art, preferably with para-aminobenzoic acid (PABA) derivatives (e.g., butyl-PABA) or 2-aminoacridone. The fragmentation of derivatives is investigated to determine the most specific and sensitive scan function for TMS. For example, the present investigators have determined that butyl-PABA derivatives of (Glc)₄ may be detected by following the transition of m/z 866 to m/z 509 by multiple reaction monitoring using electrospray ionization-TMS (ESI-TMS) with a triple quadrupole mass spectrometer. Those skilled in the art will appreciate that other derivatization methods may be used, and appropriate scan functions may be used to detect these alternative (Glc)₄ derivatives. Likewise, numerous alternative ionization methods are known in the art (e.g., Matrix Assisted Laser Desorption Ionization; MALDI) as alternatives to ESI.

In particular embodiments, the analyte is concentrated prior to TMS analysis, as described hereinabove. It is particularly preferred that the (Glc)₄ is concentrated by immunoprecipitation with paramagnetic beads to which an antibody that specifically recognizes (Glc)₄ is conjugated. The (Glc)₄ may then be eluted from the beads using an appropriate solvent. Typically, but not necessarily, the concentration step is carried out prior to derivatization. In other particular preferred embodiments, other analytes of interest may be immunopurified from the same sample. For example, a mixed population of beads, each carrying antibodies that are specific for a different analyte that is characteristic of a metabolic disorder, may be added to the sample. In this manner, the same sample may be used to screen for multiple inherited metabolic disorders.

Alternatively, the (Glc)₄ may be concentrated using methods based on size exclusion.

Further, a “clean-up” or pretreatment step may be employed to reduce or remove interfering or otherwise undesirable substances. For example, if the ratio of glucose to (Glc)₄ in the biological sample is relatively high (e g., 2:1 or higher), it is preferable to reduce the glucose concentration in the samples prior to analysis by TMS. The concentration of glucose in the sample may be reduced by any method known in the art. One exemplary, and preferred, method is to subject the sample to enzymatic treatment to remove glucose, typically prior to derivatization. For example, the sample may be digested with glucose oxidase to reduce or remove glucose from the biological sample. The enzymatic treatment should preferably not degrade the (Glc)₄ tetrasaccharide, or only do so to an insignificant extent. Alternatively, glucose may be separated from (Glc)₄ tetrasaccharide using separation (e.g., chromatographic) techniques, generally following the derivatization step. To illustrate, following the derivatization step, the derivatized glucose may be separated from the derivatized (Glc)₄ using liquid chromatography (e.g., reversed phase).

An internal standard is generally added to the sample prior to manipulations, so that the standard is subjected to the same conditions as the analyte. Any suitable internal standard may be used. (Glc)₄ homologs in which one of the glucose residues is replaced by a [U-¹³C] labeled glucose to provide a mass shift of +6 Da (as described in the Examples) are suitable and preferred. The internal standard is added to the sample in a known quantity. The ratio of signals produced by (Glc)₄ in the sample and the internal standard will allow the starting quantity of (Glc)₄ in the sample to be determined by use of a calibration curve. The calibration curve is a plot of the signal ratio ((Glc)₄ to internal standard) against different known concentrations of Glc₄ standard, using the same fixed quantity of internal standard.

An alternative preferred internal standard is a deuterium labeled glucose tetramer.

A preferred method of the invention for quantifying or determining (Glc)₄ in a biological sample comprises: (1) collecting a biological sample; (2) adding a known quantity of a suitable stable isotope-labeled standard to the sample; (3) optionally, concentrating the (Glc)₄ in the sample by immunoprecipitation with magnetized beads, followed by elution from the beads with a suitable solvent; (4) derivatization of the glycans, e.g., with butyl-PABA or 2-aminoacridone; and (5) quantification of the (Glc)₄ using TMS. Optionally, interfering glucose signals may be reduced by enzymatic treatment prior to step 4 or by chromatographic separation prior to step 5, as described above.

Preferably, a [U-¹³C] labeled glucose tetramer is used as an internal standard for the TMS analysis.

The foregoing methodology may be employed in preferred embodiments of the inventive screening and monitoring assays described above. As further described above, it is preferred that the methods be partially or completely automated.

TMS based methodologies are particularly suitable for quantifying or determining (Glc)₄ in dried blood spots from neonatal screening cards. According to this embodiment, the method above further comprises a step of extracting oligosaccharides from the dried blood spot using a suitable solvent (e.g., an aqueous solvent or aqueous/organic mixture). Alternatively, TMS may be used to quantify or determine the presence of (Glc)₄ in dried urine samples (e.g., on filter papers or diaper material).

Thus, as a particularly preferred embodiment, the present invention provides a method of screening a neonatal subject for Pompe disease, comprising: (1) providing a blood sample, typically in the form of a dried blood spot on a neonatal screening card (e.g., a filter paper); (2) extracting oligosaccharides from the dried blood spot using a solvent; (3) adding a known quantity of an appropriate stable isotope-labeled internal standard to each sample; (4) derivatizing the oligosaccharides (e.g., with butyl-PABA); (5) analyzing the (Glc)₄ derivatives by TMS using a specific scan function; (6) quantifying or determining the (Glc)₄ in the sample by comparing the signal produced by the derivatized (Glc)₄ with the signal produced by the derivatized internal standard; and (6) presumptively identifying those subjects as affected with Pompe disease based on (Glc)₄ concentrations in the sample that are greater than a reference value (as described above).

This method may optionally further comprise analyte concentration steps and glucose removal steps as described hereinabove.

Having now described the invention, the same will be illustrated with reference to certain examples, which are included herein for illustration purposes only, and which are not intended to be limiting of the invention.

### EXAMPLE 1

### Material and Equipment

α-D-Glc(1→6)-α-D-Glc(1→4)-α-D-Glc(1→4)-D-Glc {(Glc)₄}, maltotetraose (M₄), maltopentaose (M₅), maltohexaose (M₆), maltoheptaose (M₇), cellopentaose (C5), sodium cyanoborohydride (NaBH₃CN), benzoic anhydride, Butyl-4-aminobenzoate (BAB), 1-phenyl-3-methyl-5-pyrazolone (PMP), and 2′-fucosyllactose were purchased from Sigma-Aldrich (St. Louis, Mo.). 2-Aminoacridone (AMAC) was from Molecular Probes, Inc. (Eugene, Oreg.). Methanol, acetonitrile (HPLC grade), acetic acid and hydrochloric acid were purchased from VWR Scientific products (Atlanta, Ga.). All other reagents were of analytical grade and commercially available.

All HPLC solvents were filtered (0.2 μm membrane) and degassed just prior to use. PMP was recrystallized from methanol prior to use. Sep-Pak® Vac C18 cartridges (100mg) and YMC-Pack Pro C₁₈ column (250×4.6 nm I.D., 5 μm) were purchased from Waters (Franklin, Mass.). The HPLC system was equipped with Waters 626 pump, 486 tunable absorbance detector, 717 plus autosampler, and 600S controller (Waters, Milford, Mass.). Mass spectral analysis was performed on a Quattro-LC electrospray ionization triple quadrupole tandem mass spectrometer (ESI-MS/MS), (Micromass Inc., Beverly, Mass.)), (Micromass Inc., Beverly, Mass.) equipped with a Hewlett-Packard binary pump and Gilson 215 liquid handler. Lyophilized recombinant TVA II used for the synthesis of the internal standard was the generous gift of Dr. Takashi Tonozuka (Department of Applied Biological Science, Tokyo University of Agriculture and Technology, Tokyo).

### EXAMPLE 2

### HPLC Assay for (Glc)₄

Sample Preparation for HPLC Analysis

For samples isolated from patients on enzyme replacement therapy, plasma and 6 h urine samples were collected before the initiation of the therapy and every 2 weeks during the therapy. Both urine and plasma samples were frozen at −20° C. before testing for (Glc)₄ levels.

Urine samples were centrifuged and 50 μl of the supernatant was mixed with 10 μg of internal standard, cellopentaose (C5) in 10 μl of de-ionized water. Urine standards were prepared by adding known amounts of (Glc)₄ in 10 μl of de-ionized water to 50 μl aliquots of control urine containing 10 μg of C5.

Plasma or serum (200 μl) was mixed with 1 μg of internal standard (C5) and 500 μl of methanol in a glass conical test tube and centrifuged at 6,000 rpm for 4 minutes to pellet denatured proteins. The supernatant was dried under N₂ and reconstituted in 60 μl of de-ionized water. Plasma standards were prepared by adding a known amount of (Glc)₄ to 200 μl aliquots of a plasma control sample.

Derivatization of Oligosaccharides for HPLC Analysis of (Glc)₄

Oligosaccharides were derivatized with butyl-p-aminobenzoate (BAB) using a modification of the method of Poulter and Burlingame ((1990) Methods Enzymol. 193, 661-689). Derivatizing reagent, prepared freshly as required, contained BAB (54 mg), NaBH₃CN (47 mg), acetic acid (0.11 mL), and methanol (1.76 mL). To each sample, prepared as described above, were added 140 μl of the reagent. The sample mixtures were incubated at 80° C. for 45 min and then cooled to room temperature. 0.9 ml of 15% acetonitrile was added and the mixture was vortexed for 10 s. Solid phase extraction was used to remove unreacted reagent from the derivatized oligosaccharides. Samples were loaded onto a C₁₈ cartridge preconditioned with 1 ml methanol followed by 1 ml de-ionized water and washed with 1 ml 15% v/v acetonitrile/water. The BAB-labeled oligosaccharides were then eluted with 1 ml 30% v/v acetonitrile/water. For urine samples, the eluate was directly analyzed by HPLC. For plasma samples, the eluate was dried under N₂ and reconstituted in 150 μl 30% v/v acetonitrile/water prior to analysis.

2-Aminoacridone (AMAC), PMP, and perbenzoyl (PB) derivatives of oligosaccharides were prepared according to the published procedures (Okafo, G., et al. (1996) Anal. Chem. 68, 4424-4430; Zopf, D., and Fu, D., (1999) Anal. Biochem. 269, 113-123; Daniel, P. F., et al. (1981) Carbohydr. Res. 97, 161-180).

HPLC Analysis and Quantitation

The BAB-labeled oligosaccharides were separated on a YMC-Pack Pro C₁₈ column at a flow rate of 0.5 ml/min and UV absorbance of the effluent was monitored at 304 nm. The HPLC elution was isocratic for 30 min with 30% acetonitrile and 70% 0.01 mM tetrabutylammonium chloride, adjusted to pH 4-6 using 6N HCl. Excess unreacted BAB and other impurities were washed from the column by increasing acetonitrile to 50% at 32 min and returning to initial conditions at 38 min. Total analysis time was 45 min per sample. The peak areas of (Glc)₄ and the internal standard (C5) were calculated automatically with baseline correction where appropriate, and the ratio of these areas was used to quantify (Glc)₄.

ESI-MS/MS Analysis of Standards and HPLC Fractions

BAB oligosaccharide derivatives, either in whole samples or collected as fractions after HPLC separation, were analyzed by ESI-MS and ESI-MS/MS on a tandem mass spectrometer. Injection was performed via a 20 μl Rheodyne loop into a carrier solvent of acetonitrile:water (1:1; v/v) at a flow rate of 15 μl/min. The capillary and cone settings were 3.50 kV and 78-95 V, respectively, and the source block and desolvation temperatures were 80 and 150° C., respectively. A collision energy of 45-77 eV and gas cell pressure of 3.5×10⁻³ mBar were used for collision-induced dissociation experiments. Mass spectra were acquired in positive ion mode with a scan rate of 100 amu/s.

### EXAMPLE 3

### Separation and Analysis of (Glc)₄ by HPLC

Four derivatives were compared for suitability in the quantitation of oligosaccharides by HPLC using the same high-resolution liquid chromatography column described above in Example 2. They were buryl-p-aminobenzoate (BAB), 2-aminoacridone (AMAC), 1-phenyl-3-methyl-5-pyrazolone (PMP), and benzoic anhydride. BAB derivatization was ultimately selected for this application based on its advantages of sensitivity, reproducibility, high resolution, simplicity, and low cost. In this comparison, AMAC derivatization had higher sensitivity but was unable to separate (Glc)₄ from lactose found in high quantities in urine, and PMP derivatization had good resolution but a relatively lower sensitivity. Perbenzoylation had good resolution and fair sensitivity, but proved to be too time consuming. The entire procedure, including the reduction of anomers and complete perbenzoylation, took over 30 hours, compared with only 2 hr for the BAB method. Furthermore, the BAB derivatives were stable for several weeks at 4° C., whereas the PMP and AMAC derivatives were much less stable.

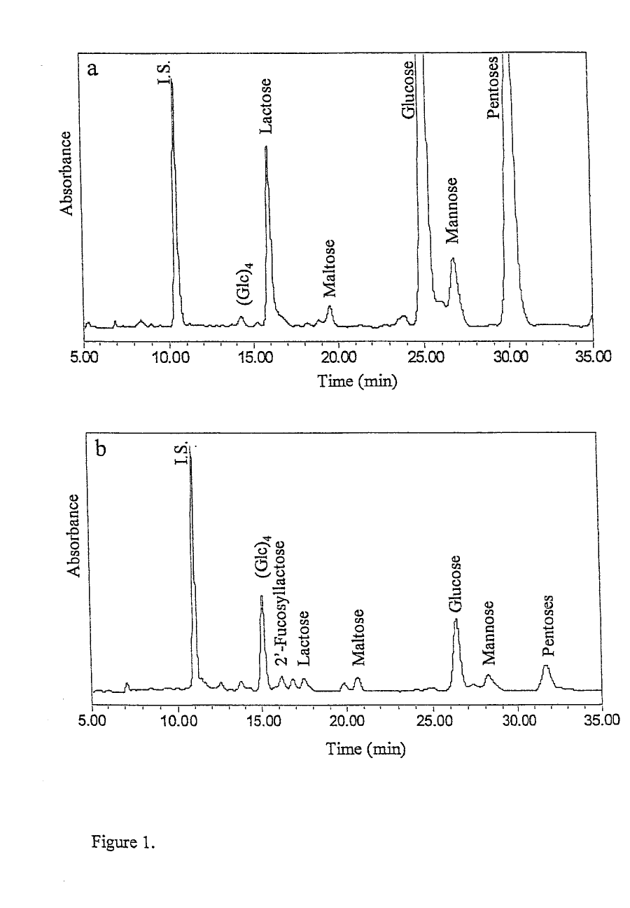

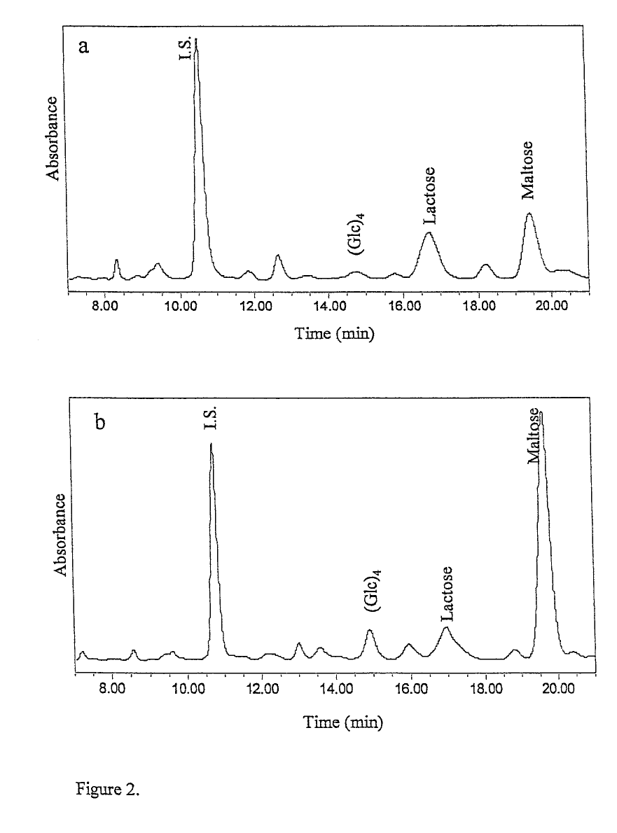

The separation of BAB-labeled (Glc)₄ from other oligosaccharides, occurring in urine and plasma, was achieved by judicious selection of the eluting solvent and flow rate and by analyzing a large number of samples from children of various ages in whom no disease was known to be present (controls). The specificity of the method was virtually guaranteed by the absence of known interfering signals at the retention time of (Glc)₄, as determined by the analysis of fractions from selected patient samples by ESI-MS/MS. An example of a normal urine chromatogram showing the separation of (Glc)₄ from other oligosaccharides is provided in FIG. 1 (panel A). A GSD-II patient's urine showing a much larger signal for (Glc)₄ is included for comparison (FIG. 1, panel B). Other identified urinary oligosaccharides are labeled in the FIG. 1 (panels A and B). It is noteworthy that the relatively low glucose signal in the patient's urine is consistent with the phenotype of hypoglycemia in Pompe disease (Chen, Y. T. and Burchell, A., (1995) in The Metabolic and Molecular Bases of Inherited Disease, 7^{th} Edition, Volume 2 (Scriver, C. R., Beaudet, A. L., Sly, W. S., and Valle, D. Eds), pp. 935-965, McGraw-Hill, New York). Comparison of plasma (Glc)₄ levels in a normal control (FIG. 2, panel A) and a patient with GSD-II (FIG. 2, panel B) were also performed. The large glucose signal was excluded for clarity.

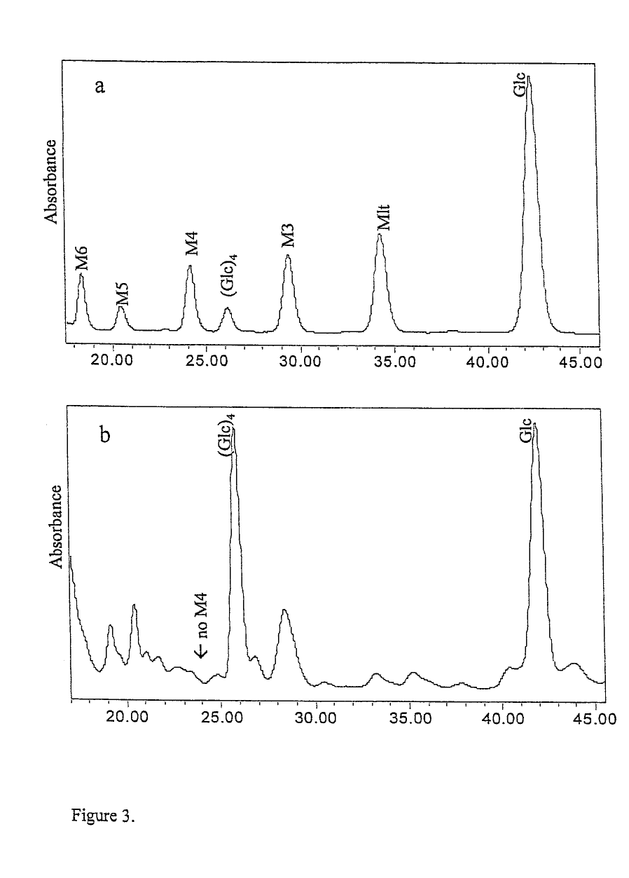

It should be noted that the BAB method cannot separate (Glc)₄ from maltotetraose (M₄). The inventors verified the absence of M₄ in selected controls and patients by analysis of PMP derivatives, which enables complete separation of M₄ from (Glc)₄, as shown by the example in FIG. 3. Based on this method, the content of M₄ in urine was estimated to be <1 μg/ml, which is below the detection limit of the BAB method and therefore considered to be negligible.

### EXAMPLE 4

### Sensitivity and Specificity of the HPLC Method for (Glc)₄

The absolute sensitivity of the method for (Glc)₄, defined as a signal-to-noise ratio of greater than three, was 3 ng (4.5 pmol) for a single HPLC injection. For a urine sample the detection limit was 1.2 μg/ml, based on the injection of 50 μl from a total sample volume of 1 ml, and for plasma the detection limit was 0.02 μg/ml, based on the injection of 100 μl from a total of 150 μl. This sensitivity is more than adequate, based on the range of normal control values (see Table 1).

TABLE I
(Glc)₄ Concentration in Urine and Plasma of Normal Controls¹

Urine (mmol/mol Creatinine)

| Age (year) | n | Mean | S.D.² | Maximum³ |
|---|---|---|---|---|
| <1 | 20 | 8.9 | 8.2 | 26.9 |
| 1-5 | 20 | 3.6 | 3.8 | 12.5 |
| 5-10 | 18 | 2 | 2.1 | 5.7 |
| 10-20 | 12 | 0.9 | 1.0 | 3.8 |
| >20 | 20 | 0.4 | 0.3 | 1.0 |

Plasma (βg/mL)

| Age (year) | n | Mean | S.D.² | Maximum³ |
|---|---|---|---|---|
| <1 | 20 | 0.2 | 0.26 | 0.37 |
| 1-5 | 20 | 0.15 | 0.10 | 0.35 |
| 5-10 | 13 | 0.15 | 0.10 | 0.36 |
| 10-20 | 11 | 0.13 | 0.10 | 0.32 |
| >20 | 12 | 0.08 | 0.06 | 0.18 |

¹ Tested for five age groups
² Standard deviation
³ Maximum (Glc)₄ concentration measured in the corresponding age group

### EXAMPLE 5

### Accuracy and Precision of the HPLC Assay for (Glc)₄

The internal standard (C5) was introduced to account for any losses incurred during sample preparation and analysis. A urine standard curve of the (Glc)₄ to C5 peak area ratios against added (Glc)₄ concentration was linear up to 15 μg/ml, corresponding to 300 μg/ml in a urine sample. A plasma standard curve was linear up to 1 μg/ml, corresponding to 7.5 μg/ml in a plasma sample. The r² values of the linear regressions were >0.999. The accuracy and precision of the method were well within acceptable limits for a clinical assay according to the replicate analysis of calibrators (Table 2). The reproducibility of the method was determined by analyzing the same quality control samples on a weekly basis. As shown in Table 2, results were in agreement within 10% for both urine and plasma control samples.

TABLE 2
Interday Accuracy and Precision of (Glc)₄ Assay
According to Calibrators

| Urine (n = 4) | | | | Plasma (n = 4) | | | |
|---|---|---|---|---|---|---|---|
| True | Mean | cv | error | True | Mean | cv | error |
| (μg) | (μg) | (%) | (%) | (μg) | (μg) | (%) | (%) |
| 0.5 | 0.53 | 9.34 | 5.2 | 0.05 | 0.05 | 8.7 | −2.56 |
| 1.0 | 1.02 | 7.13 | 1.48 | 0.1 | 0.11 | 7.76 | 6.98 |
| 2.5 | 2.57 | 4.52 | 2.63 | 0.2 | 0.21 | 6.58 | 5.55 |
| 7.5 | 7.38 | 2.42 | −1.59 | 0.5 | 0.50 | 3.5 | −0.15 |
| 15 | 15.02 | 1.91 | 0.13 | 1.0 | 1.01 | 2.9 | 1.38 |

According to Quality Controls

| Urine (μg) | | | | | | | |
|---|---|---|---|---|---|---|---|
| Low QC | | | | Plasma (μg) | | | |
| (n = 12) | | High QC (n = 12) | | Low QC (n = 4) | | High QC (n = 10) | |
| Mean | cv (%) | Mean | cv (%) | Mean | cv (%) | Mean | cv (%) |
| 0.83 | 8.3 | 5.82 | 5.9 | 0.076 | 10.1 | 0.6 | 9.7 |

cv (%): 100% standard deviation/mean
error (%): 100% mean error/mean

### EXAMPLE 6

### Identification of HPLC-Isolated Oligosaccharides by ESI/MS/MS

ESI-MS was employed to confirm the identity of (Glc)₄ in selected patient urine samples when the HPLC chromatographic separation was thought to be adequate. The fractions corresponding to (Glc)₄, collected during HPLC analysis of patient and control samples, were analyzed by ESI-MS. Most were found to be homogeneous for tetraglucose, as determined by the dominance of an ion mass of m/z 866 which corresponds to the sodium adduct of a BAB-labeled glucose tetramer. During method development it was observed that the amount of (Glc)₄ in a number of infant control urine samples was higher than expected when analyzed by HPLC. Analysis of the (Glc)₄ fraction by ESI-MS revealed that it co-eluted with a compound of m/z 688. Using ESI-MS/MS analysis, this compound was shown to be the sodium adduct of a deoxyhexose-hexose-hexose PAB derivative. The supposition that this compound was 2′-fucosyl-lactose, a component of human breast milk (Chaturvedi, P, et al. (1997) Anal. Biochem. 251, 89-97), was confirmed by HPLC and ESI-MS/MS analysis of an authentic specimen. The HPLC method was then modified appropriately to resolve this compound from (Glc)₄. This underscores the value of mass spectrometry in the development of clinical HPLC assays dependent on detectors that are not molecularly-specific.

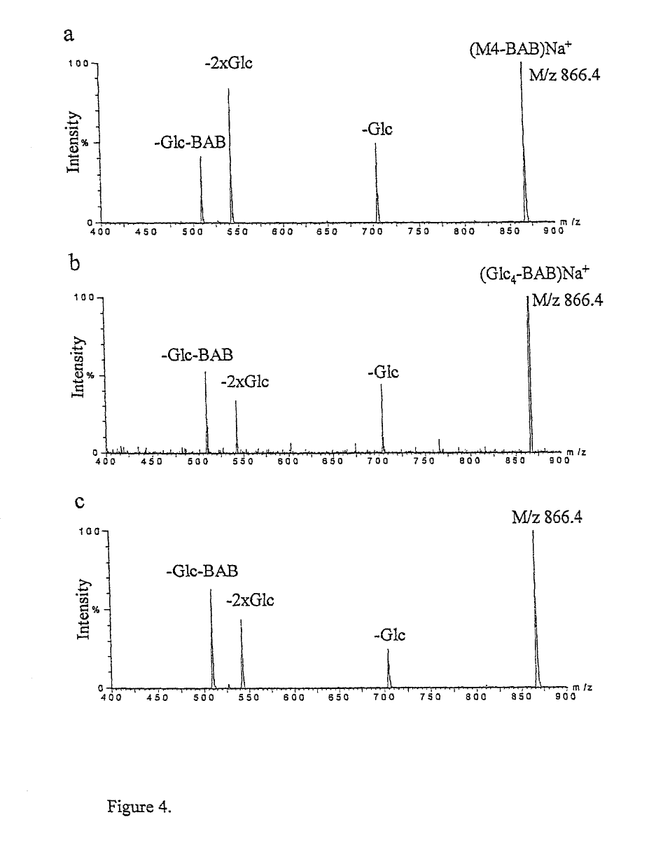

ESI-MS/MS was also employed to differentiate (Glc)₄ from the isomer maltotetraose (M₄), because these compounds were not separated by HPLC under the assay conditions. Collision-induced dissociation (CID) of the Na⁺ adducts of (Glc)₄ and M₄ results in the fragmentation patterns shown in FIG. 4. The ions at m/z 704 and 542 arise by successive losses of glucose residues from the non-reduced-end with sodium cation retention on the PAB-modified glucose residue, whereas the ion at m/z 509 arises from loss of PAB-glucose residue with sodium cation retention on the non-reduced-end. The mean (±2 standard deviation (SD)) intensity ratio of fragment m/z 509 to m/z 542 was determined to be 1.57 (±0.11) in the (Glc)₄ spectrum (FIG. 4, panel A), and 0.65 (±0.05) in the M₄ spectrum (FIG. 4, panel B). The error in the ratios was found to be 3.6% for (Glc)₄ and 4.3% for M₄ for seven replicate analyses performed over a period of three weeks. These data imply that residue losses from the reduced-end is favored over losses from the non-reduced-end in (Glc)₄, whereas for M₄ the opposite appears to be true. The ratio of the m/z 509 to m/z 542 fragment ions from the hexose tetramer in the urine of six different patients was 1.64±0.44 (mean±2) which was comparable to that of the (Glc)₄ standard, indicating that the tetramer was indeed predominantly (Glc)₄ An example is shown in FIG. 4 (panel C).

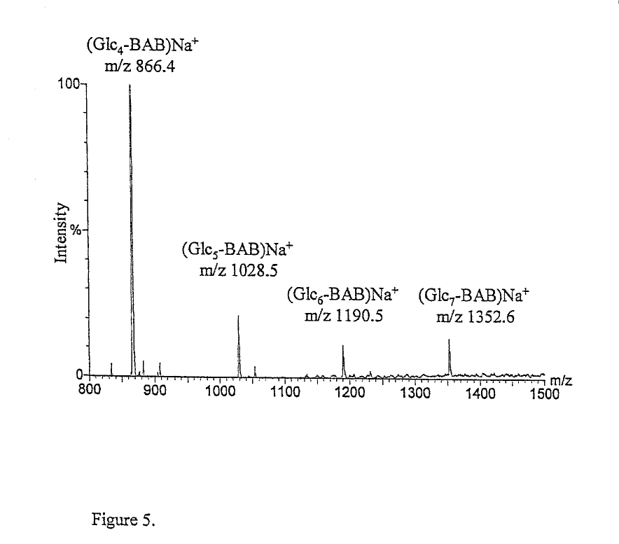

ESI-MS/MS was further applied to characterize the larger oligosaccharides seen in the urine of some patients with GSD-II. An example of the ESI-MS analysis of total BAB-derivatized urine from such a patient is shown in FIG. 5. The identities assigned to the ions of m/z 866, 1028, 1190 and 1352 are the sodium adducts of hexose oligomers having 4, 5, 6 and 7 units respectively. The signals for these ions are much lower or absent in control urine samples and it was inferred that they are all derived from glycogen. Analysis of these adducts using ESI/MS/MS revealed product ions with identical masses to those derived from the standards M₅, M₆ and M₇, confirming that they are hexose oligomers. However, as with (Glc)₄ and M₄, there were differences in the intensities of certain ions, and these are summarized in Table 3. The major differences between the urinary hexose oligomers and M₅, M₆, and M₇ are the ratios of m/z 509 to m/z 542, m/z 671 to m/z 704 and m/z 833 to m/z 866, respectively. These data indicate that losses from the reduced-end were favored in the urinary hexose oligomers as determined by the higher intensity of the product ions from this fragmentation pathway relative to the product ions derived from the non-reduced-end. These results imply that the hexose oligomers in the patient urine include at least one α-1→6 linkage, as reported previously for glucose oligomers, containing 6 to 8 residues, identified in the urine of patients with GSD-II and GSD-III (Lennartson, et al. (1978) Eur. J. Biochem. 83, 325-334).

TABLE 3
Fragment Intensity Ratios of BAB-Labeled Maltoseries Standards and
Hexose Oligomers Present in the Urine of GSD-II Patients
Analyzed by ESI-MS-MS

| | m/z Ratio | Intensity | | Intensity |
|---|---|---|---|---|
| Oligosaccharides | A | Ratio | m/z Ratio B | Ratio |
| M5 | 671/866 | 0.78 | 671/704 | 0.34 |
| Hexose pentamer | 671/866 | 1.7 | 671/704 | 0.96 |
| M6 | 833/1028 | 0.69 | 833/866 | 0.50 |
| Hexose hexamer | 833/1028 | 3.0 | 833/866 | 1.7 |
| M7 | 995/1190 | 0.65 | 995/1028 | 1.0 |
| Hexose heptamer | 995/1190 | 1.2 | 995/1028 | 1.94 |

Loss of the BAB-labeled hexose at the reduced end results in fragment ions m/z 671, 833, and 995 of the hexose pentamers, hexamers, and heptamers, respectively. The ratios of these ions to those produced from:
A. the loss of one residue from the non-reduced end (m/z 866, 1028, and 1190, respectively),
B. the loss of two residues from the non-reduced end (m/z 704, 866, and 1028, respectively) are shown.
Ratios are the mean of 3 replicate injections for the maltoseries standards and of 2 different patient samples for the urinary oligomers.

### EXAMPLE 7

### Concentration of (Glc)₄ in Urine and Plasma

The (Glc)₄ concentrations in urine and plasma of normal controls, separated by age range, are summarized in Table 1. An inverse relationship of (Glc)₄ excretion with increased age was observed, which was quantitatively more evident in the urine than in the plasma. The (Glc)₄ concentrations in the urine and plasma of patients with GSD-II also appeared to be age-dependent as shown in Tables 4 and Table 5.

TABLE 4
(Glc)₄ Levels in Urine of Glycogen Storage Disease Patients

| | Age | (Glc)₄ | (Glc)₄² (Normal Range) |
|---|---|---|---|
| Status | (Year) | (mmol/mol Cr¹) | (mmol/mol Crhu 1) |
| GSD II | 0.1 | 344 | |
| GSD II | 0.2 | 45.5 | |
| GSD II | 0.5 | 45.6 | |
| GSD II | 0.5 | 31.5 | 8.9 ± 8.2 |
| GSD II | 0.8 | 17.6 | |
| GSD II | 0.9 | 33.1 | |
| GSD II | 2.5 | 54.7 | |
| GSD II | 3.0 | 52.2 | |
| GSD II | 4.0 | 27.2 | 3.6 ± 3.8 |
| GSD II | 4.0 | 92.8 | |
| GSD II | 5.5 | 73.4 | |
| GSD II | 11 | 31.0 | 2.0 ± 2.1 |
| GSD II | 20 | 33.0 | |
| GSD II | 31 | 25.0 | |
| GSD II | 40 | 2.2 | |
| GSD II | 45 | 4.8 | 0.4 ± 0.3 |
| GSD II | 45 | 8.8 | |
| GSD II | 61 | 6.5 | |
| GSD Ia | 2 | 4.8 | 3.6 ± 3.8 |
| GSD Ia | 6 | 4.8 | |
| GSD Ib | 19 | 0.8 | 2.0 ± 2.1 |
| GSD IIIa | 4 | 18.2 | |
| GSD III | 5 | 97.6 | 3.6 ± 3.8 |
| GSD IIIb | 9 | 23.9 | |
| GSD IIIa | 28 | 4.8 | |
| GSD III | 29 | 1.8 | 0.4 ± 0.3 |
| GSD IIIb | 46 | 2.1 | |

¹ Cr = Creatinine
^{2 Urine (Glc)}₄ levels (average ± standard deviation) of normal individuals in the corresponding age groups
TABLE 5

(Glc)₄ Levels in Plasma of Glycogen Storage Disease Type II Patients

| Age | (Glc)4 (μ/mL) | (Glc)41 (Normal Range) (μ/mL) |
|---|---|---|
| 0.1 | 0.7 | |
| 0.2 | 1.19 | |
| 0.5 | 1.13 | 0.2 ± 0.26 |
| 0.8 | 4.8 | |
| 0.5 | 2.24 | |
| 0.9 | 2.0 | |
| 2.5 | 0.89 | |
| 2.5 | 2.16 | |
| 3.0 | 1.47 | 0.15 ± .01 |
| 4.0 | 0.51 | |
| 4.0 | 2.15 | |
| 5.5 | 0.37 | 0.15 ± 0.1 |
| 20 | 0.87 | |
| 31 | 0.67 | |
| 40 | 0.12 | |
| 44 | 0.66 | 0.08 ± 0.06 |
| 45 | 0.19 | |
| 45 | 0.17 | |
| 61 | 0.08 | |

¹Plasma (Glc)₄ levels (average ± standard deviation) of normal individuals in the corresponding age groups.

It has previously been reported that excretion of (Glc)₄ in urine is affected by diet, fasting status, and physical activity (Walker, G. J. and Whelan, W. J. (1960), Biochem. J. 76, 257-263). The urinary (Glc)₄ levels in Table 1 were normalized to urinary creatinine concentrations. No attempt was made to control for the factors of diet and physical activity during sample collection. However, results from 21 patients with GSD-II (infantile, childhood, and adult forms) showed that the (Glc)₄ concentrations in both plasma and urine are consistently higher, by at least a factor of 2, than those of age-matched normal controls. Table 4 also shows the urine (Glc)₄ concentration for some patients with GSD-I and GSD-III. The patients with GSD-III accumulate glycogen and excrete elevated levels of (Glc)₄. It has been shown in vitro that (Glc)₄ is a limit dextrin resulting from α-amylase degradation of glycogen (Walker, G. J. and Whelan, W. J. (1960), Biochem. J. 76, 257-263, Ugorski, M., et al. (1983) J. Exp. Pathol. 1, 27-38). Intravenous administration of glycogen in a Rhesus monkey was shown to increase (Glc)₄ excretion (Kumlien, J., et al. (1988) Clin. Chim. Acta 176, 39-48). It was reported that acid α-glucosidase (GAA) degrades glycogen in both 1→4 linkage and 1→6 linkage (branching site) (Brown, B. I. et al. (1970) Biochem. 9, 1423-1428). Therefore, the glycogen accumulated due to GAA deficiency (GSD-II) and de-branching enzyme deficiency (GSD-III) may have a similar configuration. An increase in glycogen release into the circulation, due to breakdown and turnover of glycogen-laden tissues, would be expected to increase the (Glc)₄ concentration in plasma and urine. The concentrations of (Glc)₄ in urine of patients with GSD-I are within the control range. This is likely due to the fact that the predominantly-stored material in GSD-I is fat rather than glycogen (Chen, Y. T. and Burchell, A., (1995) in The Metabolic and Molecular Bases of Inherited Disease, 7^{th} Edition, Volume 2 (Scriver, C. R., Beaudet, A. L., Sly, W. S., and Valle, D. Eds), pp. 935-965, McGraw-Hill, New York).

### EXAMPLE 8

### Application of the HPLC Method for Clinical Diagnosis

The plasma (Glc)₄ levels in GSD II patients are reported here for the first time. All infantile onset and juvenile onset patients have markedly elevated levels, whereas two patients with adult-onset GSD II were within normal range. The urine (Glc)₄ concentration measured for GSD II patients in this laboratory ranged from 19 to 821 nmol/mg creatinine (2.2-92.8 mmol/mol creatinine), which is comparable with the range of values from five GSD-II patients (6.8-908 nmol/mg creatinine) reported by Oberholzer and Sewell ((1990) Clin. Chem. 36, 1381). The values (78-324 mmol/mol creatinine) reported by Peelen et al ((1994) Clin. Chem. 40, 914-921) are much higher. Lundblad and co-workers have reported (Glc)₄ concentrations as excretion rates in mg/24 hours ((1976) Biomed. Mass Spectrom. 3, 51-54). This should arguably provide the most accurate data on the status of (Glc)₄ accumulation, but 24 hour urine sample collections are usually not practicable in a clinical diagnostic setting. Based on the results obtained in this study, a spot urine sample (with known creatinine level), from a patient who is at a high clinical suspicion or risk of having Pompe disease, should suffice to make a presumptive diagnosis within 24 hr of sample receipt, provided that the appropriate control age range is used for comparison. A muscle biopsy or skin fibroblasts would then be recommended to confirm the disease diagnosis by classical enzymology.

### EXAMPLE 9

### Use of (Glc)₄ as a Biomarker

Study Subjects

Three infants affected with infantile GSD-II as evidenced by reduced GAA activity to less than 1% of normal in skin fibroblasts and/or muscle were enrolled in the study for a phase I/II clinical trial of enzyme replacement therapy. The enzyme source was recombinant human GAA (rhGAA) purified from the culture medium of rhGAA secreting CHO cells. The study was approved by the institutional review board and parental written informed consent was obtained. Detailed patients characteristics and clinical evaluation of cardiac, pulmonary, neurologic and motor functions were described in the paper reporting clinical results (Amalfitano A, et al. (2001) Genet. Med. 3:132). These three patients had different clinical presentation in terms of severity at the initiation of the therapy. Patient one (Pt1) was treated at 4 months of age and had the most advanced stage of the disease with massive cardiomegaly, severe motor delay, and feeding difficulty with failure to thrive. Pt 2 began the treatment at 3 months of age, had severe cardiomyopathy, moderate motor delay and feeding difficulty. Pt3 was at early stage of the disease when the treatment started at 2½ months of age. He had significant motor delay but no cardiomyopathy.

(Glc)₄ Measurement

Plasma and 6 hrs urine were collected before the initiation of the therapy and every 2 weeks during the therapy. Both urine and plasma samples were frozen at −20° C. before testing. The (Glc)₄ levels in urine and plasma were measured by HPLC as described above in Example 2 using BAB as a derivative. For qualitative analysis, C5 was added to each tested sample as internal standard, and two control samples (low and high (Glc)₄ contents) were measured daily.

Monitoring of Disease Progression and Response to Therapy Using the HPLC Method

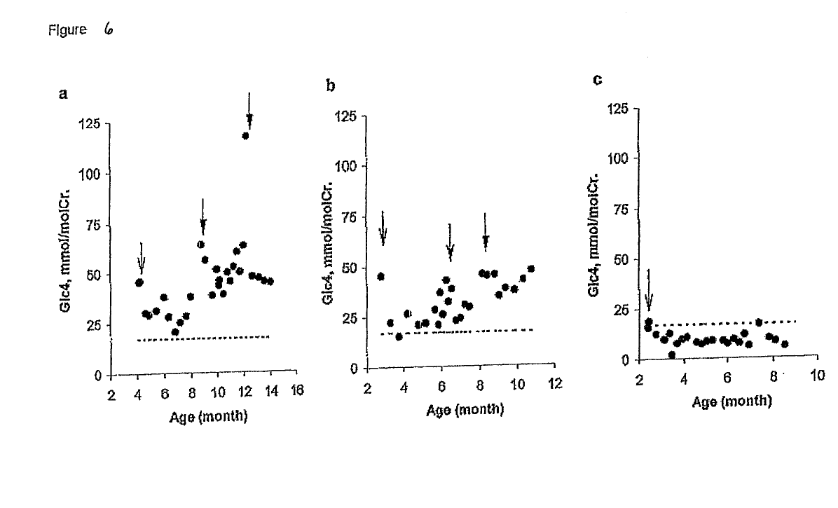

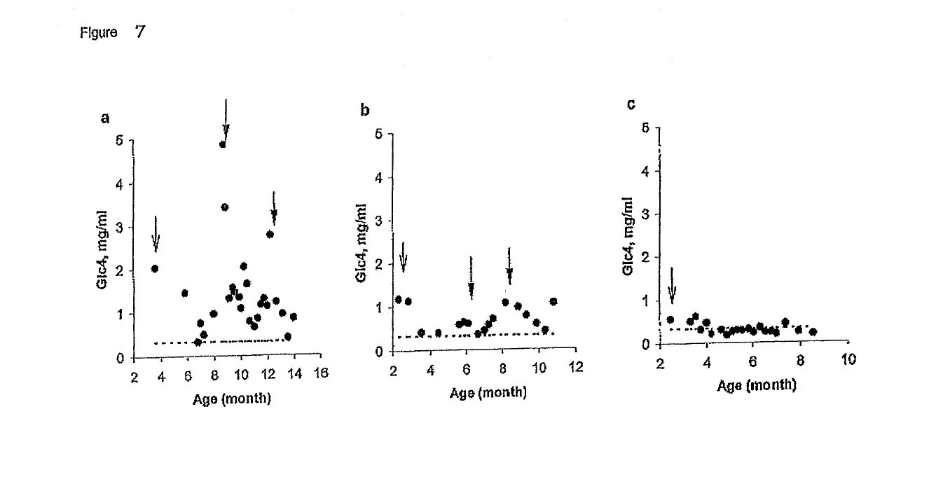

The urinary (Glc)₄ levels for the three patients before and during treatment are shown in FIG. 6. The (Glc)₄ levels in plasma are shown in FIG. 7. The (Glc)₄ levels correlated well with the clinical severity at the initiation of the therapy. Pt 1 who had the most advanced stage of the disease also had the highest (Glc)₄ levels in both plasma and urine, while Pt 2 had moderate severity of the disease and intermediate increased levels of (Glc)₄ content. Pt 3 had mildest disease symptoms and had (Glc)₄ elevation only in the plasma and was normal in the urine. It appears that as a biomarker for Pompe disease, (Glc)₄ measurement in plasma is more sensitive than its level in urine. This is reflected by a higher magnitude of (Glc)₄ elevation in the plasma than the levels in the urine and normal urine level in Pt 3.

The (Glc)₄ levels decreased in both urine and plasma for all three patients, during the first 2-3 months of therapy (FIGS. 6 and 7). Again the decrease is more striking in the plasma than in the urine. However, the (Glc)₄ levels in Pts 1 and 2 rose subsequently and concomitantly with clinical decline and the production of antibody against the rhGAA. An attempt to remove antibody and induce tolerance with plasmaphresis, IVIG, cytoxan and daily enzyme infusions for 10 days resulted in transient clinical improvement but subsequent clinical decline again and necessary second immune-tolerance therapy in Pt 1. There was a correlation of the (Glc)₄ levels and the clinical course and response to immune-tolerance therapy for Pt 1 (FIGS. 6 and 7). A similar correlation of clinical course and response to the immune-tolerance therapy was seen in Pt 2. Pt 3 who has not developed anti-rhGAA antibody continues to have normal cardiac, neurological and motor evaluations. The (Glc)₄ levels for Pt 3 were normalized during the first 2 months of therapy and remained largely normal since. The correlation of clinical course and (Glc)₄ levels in all three patients were again more striking with the plasma than the urine. The above results strongly suggest that the (Glc)₄ levels, particularly plasma, in Pompe disease patients are indicative of the clinical state of the disease. Measurement of (Glc)₄ in the blood appears to offer a non-invasive way of assessing overall disease state and therapeutic response to the therapy in Pompe disease, thus avoiding muscle biopsy.

### EXAMPLE 10

### Synthesis of a Stable Isotope-Labeled

### Internal Standard for (Glc)₄ Analysis by Tandem Mass Spectrometry

A stable, isotope-labeled internal standard was synthesized using the method of Tonozuka and coworkers (Tonozuka et al., (1994) Carbohydrate Research 261:157; Tonozuka et al., (1996) J. Appli. Glycosci. 43:95). In this method, pullulan, a polymer of the trisaccharide panose (Glcα1-6Glcα1-4Glc), is digested by the α-amylase, TVA II, in the presence of [U-¹³C]glucose. TVA II catalyzes transglycosylation of the panose product, adding [U-¹³C]glucose in both α1-4 and α1-6 linkage, thus resulting in the formation of glucose tetramers with a labeled residue at the reducing end.

One-hundred mg pullulan and 100 mg [U-¹³C₆]glucose were dissolved in 2 ml 50 mM sodium citrate, pH 6.0, mixed with 8 mg TVA II in 100 μl NaHPO₄ buffer, pH 6.9 and incubated at 40° C. for 5.5 hours. The mixture was cooled on ice and centrifuged through Amicon Centrifree 30 kDa molecular weight cut-off filters at 2000 g for 2 hours. The filtrate was fractionated on a gel filtration column (170×15 cm) packed with Toyopearl HW-40S, 30 μm particle size (Sulpeco, Bellefonte, Pa.) and eluted with dH₂O at a flow rate of 0.5 ml/min. Aliquots of fractions were mixed with 15 mM ammonium acetate in 65:35 acetonitrile:H₂O and analyzed by electrospray ionization-mass spectrometry (ESI-MS) using 3.5 kV capillary and 31 V cone settings, with acetonitrile:H₂O (1:1, v/v) as the mobile phase. Fractions containing [¹³C₆]-labeled hexose tetramers were pooled and dried under vacuum at 40° C. for 6 hours using a Centrivap (Labconco). The [¹³C₆]-labeled hexose tetramers were reconstituted in H₂O. The combined concentration of the [¹³C₆]-labeled hexose tetramers was determined using ESI-MS by comparison of the intensity of [M+Na]⁺ of BAB-derivatized internal standard (m/z 872) to that of unlabeled BAB-derivatized Glc₄ standard (m/z 866). The purity of the IS was determined by analysis of the BAB derivatives using HPLC with UV detection. The derivatized oligosaccharides were separated on a YMC-Pack Pro C₁₈ column (250×4.6 mm I.D., 5 μm) with gradient elution from 5:22:73 (v/v/v) 0.05 mol/L ammonium acetate: acetonitrile: H₂O, to 5:60:35 (v/v/v) 0.05 mol/L ammonium acetate: acetonitrile:H₂O (v/v) over 36 minutes. Oligosaccharides were identified by comparison of retention times to authentic standards wherever possible. Fractions were also collected from the HPLC separation, dried under N₂ and [M+Na]⁺ ions were analyzed by ESI-MS and ESI-MS/MS, using the same conditions as described below for plasma samples.

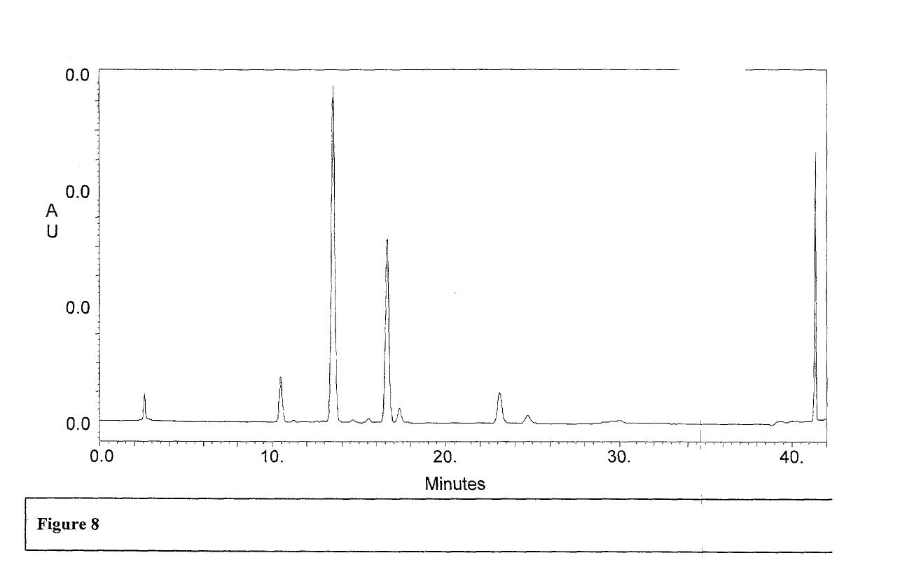

HPLC analysis of the tetrahexose fraction, isolated from the reaction mixture by gel filtration, demonstrated the presence of a number of components (see FIG. 8). Fractions collected from the HPLC analysis were analyzed using ESI-MS and ESI-MS/MS. Four components with a m/z value and fragmentation pattern expected for a BAB derivatized [¹³C₆]tetrahexose were identified (peaks 1-4 in FIG. 8). One of these components (peak 4 in FIG. 8) was identified as [¹³C₆]Glc₄ from its retention time and from the ratio of the sodiated B₃ and Y₂ fragment ions (m/z 509 and 548, respectively). As described above (Example 6: see also, An et al., (2000) Anal. Biochem. 287:136), a difference in the ratio of these two product ions for Glc₄ and maltotetraose, which has all α1-4 linkages. The ratio (mean±2SD) of m/z 509 to 548 was found to be 1.33±0.24 (n=10) for this internal standard component. For Glc₄ standard the ratio (mean±2SD) of m/z 509 to 542 (unlabeled equivalent to m/z 548) was 1.41±0.09 (n=5). Peak 2 was the major isomer present and is likely to be IMIM, which was identified by Tonozuka et al as one of the major products of the transglycosylation reaction. The ratio of m/z 509 to 548 for this isomer was 0.34±0.10 (n=10) which is similar to that of maltotetraose, which was 0.48±0.16 (n=5). The ratio for peak 1 was 1.34±0.32 (n=8). The structural identities of peaks 1 and 3 are not known and peak 3 was a mixture of a tetramer and pentamer. Panose (peak 5) and glucose (peak 6) were also present in the preparation. The three major isomers, peaks 1, 2 and 4, altogether constituted 84% of the internal standard mixture as determined by both the HPLC and MS analyses. The ratio of, peak 1:peak 2:peak 4 was 0.2:1.7:1.0.

### EXAMPLE 11

### Methods for Tandem Mass Spectrometry Analysis of Glc₄ in Plasma and Urine

Control and patient urine and plasma samples stored at −70° C. or −20° C. for up to one year were used. Normal human serum (#1101) was obtained from Biocell Laboratories, Rancho Dominguez, Calif. Urine, urine spots and plasma samples were derivatized with BAB using as previously described.

Urine: 50 μl urine was vortex mixed for 10 seconds with 25 μl 100 μmol/L internal standard and derivatized with 140 μl reagent (containing 149 mmol/L BAB, 400 mmol/L NaBH₃CN and 6% glacial acetic acid in methanol) at 80° C. for 45 minutes. It was necessary to dilute some urine samples from patients with GSD II prior to analysis by mixing 200 μl urine with 1.8 mL dH₂O.

Urine spots: Urine was centrifuged at 14 000 rpm for 5 minutes and the supernate transferred to a clean tube. Replicate 30 μL aliquots were spotted onto cotton linter paper (grade 903, Schleicher & Schuell, Keene, N. H.), and left to dry at room temperature overnight. The remaining urine was stored at −70° C. 2×¼ inch urine spots were extracted in 300 μL dH₂O by shaking at room temperature for 1 hour. 100 μL of the extract was mixed with 50 μL 2 μM IS, dried under N₂, reconstituted in 20 μL dH₂O and derivatized as above.

Plasma and serum: 100 μl plasma or serum and 50 μl 2 μmol/L internal standard were vortexed mixed with 500 μl methanol and centrifuged at 14 000 g for 5 minutes. The supernate was dried under N₂, reconstituted in 20 μl dH₂O and derivatized using 100 μl reagent (containing 400 mM BAB, 2.0 mol/L NaBH₃CN and 7.5% glacial acetic acid in methanol) at 80° C. for 45 minutes.

Derivatized urine, urine spot extract and plasma samples were purified using solid phase extraction with C18 cartridges as described above (Example 2; see also, An et al., Anal. Biochem. 287:136). The eluate was dried under N₂ at 40° C., reconstituted in 80:20 methanol:H₂O (v/v) and transferred to 96-well microtitre plates.

Calibrators: Urine calibrators were prepared using control adult urine with added Glc₄ standard ranging from 2.5 to 200 μmol/L. Urine spot calibrators were prepared with dH₂O and ranged from 0.1 to 10 μmol/L. Plasma calibrators and quality control samples were made using Biocell normal human serum, with added Glc₄ standard ranging from 0.1 to 10 μmol/L.

Mass Spectrometric Analysis and Quantitation. Urine and plasma samples were analyzed by electrospray ionisation-tandem mass spectrometry (ESI-MS/MS) using multiple reaction monitoring. (Glc)₄ and the internal standard were detected by following the transitions of m/z 866 to m/z 509 and m/z 872 to m/z 509, respectively. Urine-derived oligosaccharide derivatives were injected into 80:20 methanol:H₂O (v/v) mobile phase at a flow rate of 40 μl/min. Plasma and urine spot samples were analyzed using the same method with an additional liquid chromatography step, using a 2×100 mm C18 column (Keystone Scientific Inc.) with 80:20 methanol:H₂O (v/v) as the mobile phase at a flow rate of 200 μl/min, to concentrate the samples. Total analysis time was 2.5 minutes for urine samples and 3.0 minutes for plasma and urine spot samples. A cone voltage of 90V, capillary voltage of 3.5 kV, collision energy of 40 eV and argon collision gas pressure of 3.1×10⁻³ mBar were used. Samples were quantified using an external calibration curve derived by plotting the ratio of MRM signals for the (Glc)₄ standard to the internal standard against the concentration of added (Glc)₄.

Creatinine Measurements. Glc₄ concentrations in urine and urine spot extracts were related to the creatinine concentration. Creatinine in urine was measured using the picric acid method (Jaffe et al., (1886) Physiol. Chem. 10:391) and in paired urine and urine spot extract samples by ESI-MS/IS using a stable isotope dilution method which will be published elsewhere.

Validation of urine and plasma analysis by ESI-MS/MS. The urine and plasma analyses were validated by the replicate analysis of calibrators and quality control (QC) samples. In addition, Glc₄ concentrations in patient and control samples determined by ESI-MS/MS were compared to the results determined by HPLC-UV. Urine calibration curves and QCs were analyzed over a period of 4 weeks and plasma calibration curves and QCs were analyzed over a period of 8 weeks.

Interday variation of calibration curves and QCs for urine. The urine calibration curve was divided over two concentration ranges in order to quantify both control and patient samples where the concentration of Glc₄ may differ by one or two orders of magnitude. The interday variation of the calibration curve gradients were 1.3% for the lower range of 2.5 to 70 μM and 2.3% for the higher range of 40 to 200 μmol/L range (n=5). The mean±SD coefficient of determination (r²) of the calibration curves were 0.998±0.001 and 0.998±0.001 for the low and high ranges respectively (n=5) over 4 weeks. The interday precision and mean accuracy of calibrators are shown in Table 6. The intra- and interday precision (cv) determined by replicate analyses of a patient sample with a mean Glc₄ concentration of 31.6 mmol/mol creatinine, was 2.6% (n=5) and 5.0% (n=4) respectively. For a control sample, with mean Glc₄ of 0.4 mmol/mol creatinine, the intra- and interday precision was 4.6% (n=5) and 24.2% (n=4) respectively.

TABLE 6

| | | | |
|---|---|---|---|
| Nominal | mean | cv | |
| μmol/L | μmol/L | (%) | error % |

Low calibration range (2.5 to 70 μmol/L)

| | | | |
|---|---|---|---|
| 2.5 | 2.4 | 36.0 | 3.5 |
| 5 | 4.4 | 15.7 | 13.0 |
| 20 | 20.5 | 6.1 | −2.3 |
| 40 | 40.8 | 1.8 | −2.0 |
| 70 | 69.5 | 0.9 | 0.8 |

High calibration range (40 to 200 μmol/L)

| | | | |
|---|---|---|---|
| 40 | 40.2 | 2.4 | −0.4 |
| 70 | 69.0 | 2.2 | 1.4 |
| 100 | 100.3 | 3.0 | −0.3 |
| 150 | 151.3 | 2.3 | −0.9 |
| 200 | 199.2 | 1.1 | 0.4 |

Interday variation of calibration curves and QCs for Plasma. Plasma was quantified over 0.1 to 2.5 μmol/L and 1.0 to 10 μmol/L and the interday variation of the curve gradients were 20.3% and 20.6% (n=7) respectively over 8 weeks. The mean±SD r² value was 0.998±0.001 for the low range and 0.994±0.0009 (n=7) for the high range. The interday precision and mean accuracy of the calibrators are shown in Table 7. Plasma QCs were prepared using the same pool of normal human plasma used to prepare the calibrators. A small amount of endogenous Glc₄ was detected and determined in this plasma and accounted for in the calculations. The results for intraday and interday replicate analysis of the plasma QCs are shown in Table 8 and Table 9, respectively.

TABLE 7

| | | | |
|---|---|---|---|
| Nominal | Mean | cv | |
| μmol/L | μmol/L | (%) | error % |

Low calibration range (0.10 to 2.5 μmol/L)

| | | | |
|---|---|---|---|
| 0.1 | 0.08 | 28.0 | −18.3 |
| 0.25 | 0.26 | 8.6 | 2.5 |
| 0.5 | 0.52 | 9.5 | 3.3 |
| 1.0 | 0.98 | 8.0 | −1.9 |
| 2.5 | 2.50 | 0.9 | −0.1 |

High calibration range (1.0 to 10.0 μmol/L)

| | | | |
|---|---|---|---|
| 1.0 | 1.07 | 11.9 | 7.1 |
| 2.5 | 2.66 | 9.0 | 6.3 |
| 5.0 | 5.63 | 11.9 | 7.1 |
| 10.0 | 10.10 | 9.0 | 6.3 |

TABLE 8

| | | | cv | Mean |
|---|---|---|---|---|
| Intraday | Nominal | Mean | (%) | Error % |
| analysis | μmol/L | μmol/L | (n = 5) | (n = 5) |
| QC 1 | 0.20 | 0.18 | 21.5 | −8.5 |
| QC 2 | 1.25 | 1.30 | 7.5 | 4.2 |
| QC 3 | 8.0 | 8.71 | 1.8 | 8.9 |

TABLE 9

| | | | cv | Mean |
|---|---|---|---|---|
| Intraday | Nominal | Mean | (%) | Error % |
| analysis | μmol/L | μmol/L | (n = 7) | (n = 7) |
| QC 1 | 0.20 | 0.24 | 36.3 | 22.11 |
| QC 2 | 1.25 | 1.29 | 14.2 | 3.3 |
| QC 3 | 8.0 | 8.43 | 11.2 | 5.4 |

### EXAMPLE 12

### Comparison of ESI-MS/MS and HPLC Analyses

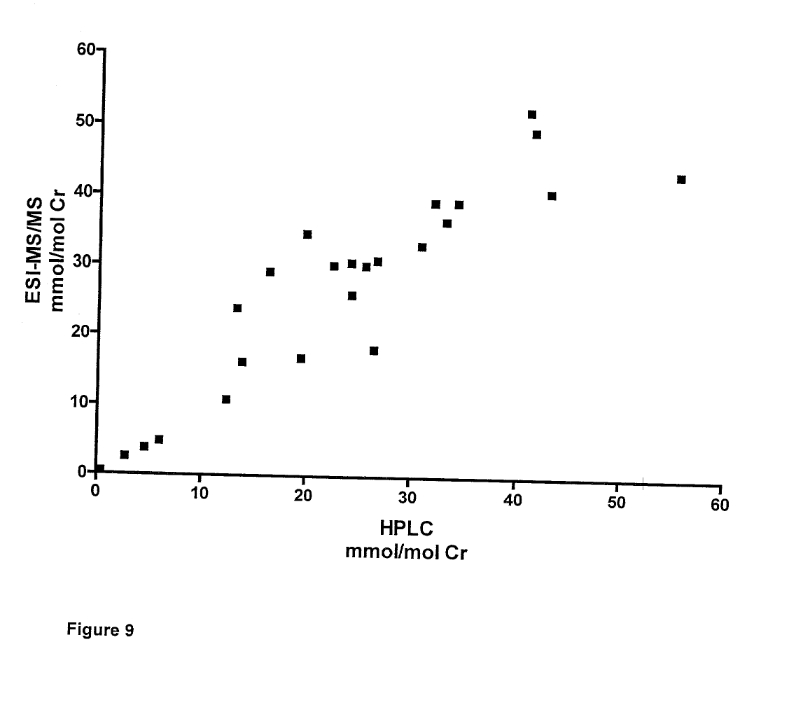

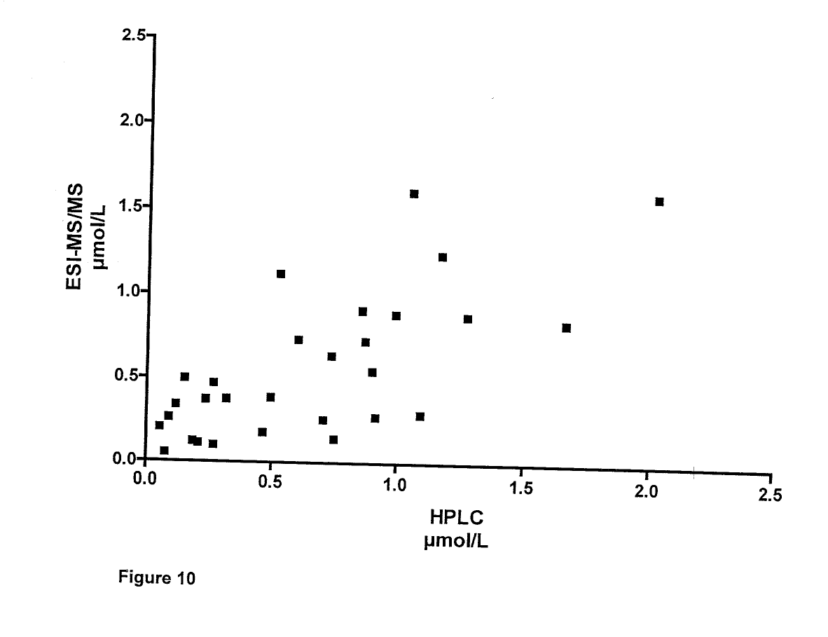

The results of 24 urine samples and 29 plasma samples (patient and controls) assayed by the HPLC method were compared with those from the ESI-MS/MS method (FIG. 9 and FIG. 10). For the urine samples y=0.97x−4.0 ; S_{y/x}=6.5 and r²=0.82. For the plasma samples y=0.62x+0.16; S_{y/x}=0.31; r²=0.502. Using Bland-Altman analysis of the data (Bland et al., (1986) Lancet 1:307), the limits of agreement [mean difference (HPLC-ESI-MS/MS)±2 SD of the difference] for urine and plasma were 3±12.4 and 0.1±0.72 respectively.

### EXAMPLE 13

### Comparison of Glc4 Analysis in Liquid and Spotted Urine Samples

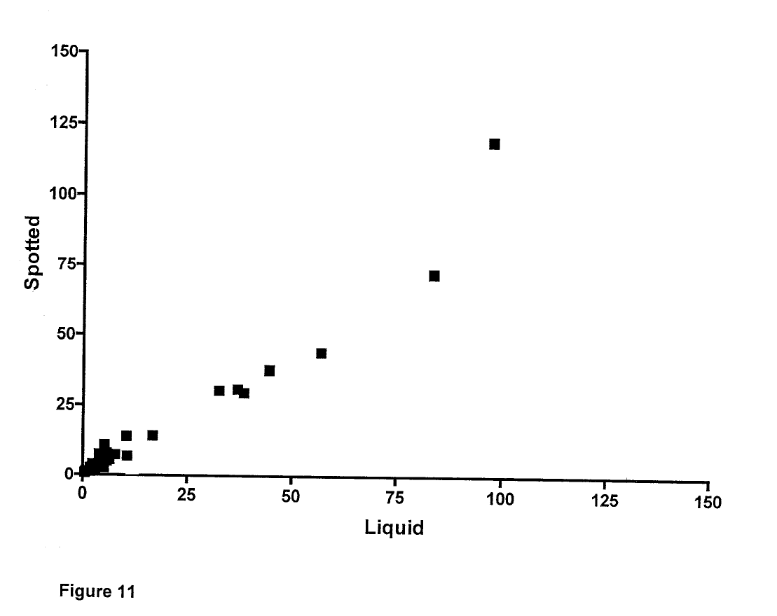

Glc₄ concentrations were determined from 37 paired liquid and spot urine samples. A comparison of the concentrations is shown in FIG. 11. y=0.99x−0.38, S_{y/x}=5.36 and r²=0.954. The limits of agreement for Bland Altman analysis [mean difference (liquid-spot)±2 SD of the difference] were 0.55±10.4.

### EXAMPLE 14

### Investigation into Possible Interferences of the Assay

A high cone voltage (90V) is used to optimize the intensity of the [M+Na]⁺ ions. At this voltage some in-source fragmentation occurs and hence there is the potential for interference with Glc₄ analysis from higher mass hexose oligomers that fragment to give a m/z 866 product ion. In order to investigate this, the extent of in-source fragmentation at different cone voltages of maltopentaose and maltohexaose was determined. In addition, the contribution of higher mass hexose oligomers in GSD II patient samples to m/z 866 was estimated. 12 μmol/L maltopentaose and maltohexaose BAB-derivatives in 80:20 methanol: H₂O (v/v) were infused into the mass spectrometer using a syringe pump (model) at a flow rate of 10 μL/minute. The cone voltage was increased from 30 to 100 V and the relative intensities of m/z 866 and [M+Na]⁺ were determined. For maltopentaose, the relative intensity of m/z 866 increased with cone voltage, from 2.5% of the intensity of m/z 1028 at 30V to 4.6% at 100 V. For maltohexaose, the relative intensity of m/z 866 did not increase with cone voltage. At 90V, the relative intensity of [M+Na]⁺ for both standards was comparable (4%). BAB-derivatives of twelve GSD II patient urine samples were analyzed in MS1 mode by scanning between m/z 830 and 1400 at a rate of 100 amu.sect⁻¹. The mean relative intensities of [M+Na]⁺ for the hexose pentamer(s), hexose hexamer(s) and hexose heptamer(s) present, to m/z 866 was determined to be 5.2, 2.8 and 3.6% respectively. Hence the combined contribution of these hexose oligomers oligosaccharides to the m/z 866 signal was determined to be 0.46%.

### EXAMPLE 15

### Neonatal Screening Assay using TMS

A TMS based assay is employed to screen neonates for Pompe disease using (Glc)₄ as a biomarker. Neonatal screening cards containing dried blood spots (typically, from heel stabs) are obtained. A disk is punched out of the blood spot and put into a vial containing solvent to extract oligosaccharides. Internal standard is added to each vial in a known quantity (e.g., a (Glc)₄ tetramer in which one of the monomers is replaced with a U-¹³C-glucose homologue). The oligosaccharides are then derivatized in the sample using butyl-PABA. The derivatized sample is analyzed by TMS as described in Example 11. The data are captured by a computer and analyzed to determine the concentration of (Glc)₄ in each sample (i.e., by comparing the ratio of signals produced by the internal standard and the analyte). Values above a reference value are indicative of Pompe disease.

Optionally, the analyte is concentrated prior to derivatization by incubating the sample with paramagnetized polystyrene spheres (Dynabeads®) with anti-(Glc)₄ Ab chemically conjugated thereto. The beads are collected magnetically, and the analyte is eluted from the beads using an appropriate solvent.

As a further optional step, the concentration of glucose monomer is reduced in the sample prior to TMS analysis. In one protocol, glucose oxidase is added to the sample prior to derivatization. In an alternate protocol, the derivatized glucose monomer is separated out by a liquid chromatography step (reversed-phase) prior to the TMS.

The foregoing examples are illustrative of the present invention, and are not to be construed as limiting thereof. The invention is described in the following claims, with equivalents of the claims to be included therein.

That which is claimed is:

1. A method of screening a subject for a glycogen storage disease, comprising the steps of: determining the concentration of hexose tetrasaccharide (Glc)₄ in a biological sample taken from the subject, and comparing the concentration to a reference value,
wherein the detection of (Glc)₄ in the biological sample at more than the reference value identifies the subject as affected with a glycogen storage disease.

2. The method of claim 1, wherein (Glc)₄ has the structure α-D-Glc(1→6)-α-D-Glc(1→4)-α-D-Glc(1→4)-D-Glc.

3. The method of claim 1, wherein the concentration of (Glc)₄ is determined using a quantitative method.

4. The method of claim 3, wherein (Glc)₄ is quantified by a method selected from the group consisting of tandem mass spectrometry, mass spectrometry, liquid chromatography, and immunopurification.

5. The method of claim 1, wherein the concentration of (Glc)₄ is determined using a semi-quantitative method.

6. The method of claim 1, wherein the glycogen storage disease is selected from the group consisting of Pompe disease (glycogen storage disease type II), glycogen storage disease type III, and glycogen storage disease type VI.

7. The method of claim 1, wherein the subject is a human subject.

8. The method of claim 7, wherein the human subject is a neonatal subject.

9. The method of claim 1, wherein the biological sample is a body fluid sample.

10. The method of claim 9, wherein the body fluid sample is selected from the group consisting of blood, plasma, serum, urine, sputum, and amniotic fluid.

11. The method of claim 10, wherein the body fluid sample is a neonatal blood sample.

12. The method of claim 11, wherein the neonatal blood sample is a dried blood spot.

13. The method of claim 9, wherein the body fluid sample is a dried urine sample.

14. The method of claim 1, wherein the biological sample is a cell or tissue sample.

15. The method of claim 1, wherein the reference value is a predetermined value.

16. The method of claim 1, wherein the reference value is based on (Glc)₄ concentrations found in a matched population of subjects.

17. The method of claim 16, wherein the matched population of subjects is an unaffected population of subjects.

18. The method of claim 1, further comprising the step of performing additional diagnostic testing on a subject that has been identified as affected with a glycogen storage disease.

19. A method of screening a subject for Pompe disease (glycogen storage disease type II), comprising the steps of: determining the concentration of hexose tetrasaccharide (Glc₄) in a biological sample taken from the subject, and comparing the concentration to a reference value;
wherein the detection of (Glc)₄ in the biological sample at more than the reference value identifies the subject as affected with Pompe Disease.

20. The method of claim 19, wherein (Glc)₄ has the structure α-D-Glc(1→6)-α-D-Glc(1→4)-α-D-Glc(1→4)-D-Glc.

21. The method of claim 19, wherein the concentration of (Glc)₄ is determined using a quantitative method.

22. The method of claim 20, wherein (Glc)₄ is quantified by tandem mass spectrometry.

23. The method of claim 22, wherein the oligosaccharides in the biological sample are derivatized with butyl-para-aminobenzoic acid prior to quantification by tandem mass spectrometry.

24. The method of claim 22, wherein the quantification by tandem mass spectrometry is standardized using a [U-¹³C]glucose labeled hexose tetramer as an internal standard.

25. The method of claim 24, wherein the internal standard comprises a [U-¹³C] labeled hexose tetramer having the structure α-D-Glc(1→6)-α-D-Glc(1→4)-α-D-Glc(1→4)-D-Glc.

26. The method of claim 19, wherein the concentration of (Glc)₄ is determined using a semi-quantitative method.

27. The method of claim 19, wherein the reference value is a predetermined value.

28. The method of claim 27, wherein the predetermined reference value is based on (Glc)₄ concentrations found in a matched population of subjects.

29. The method of claim 28, wherein the matched population of subjects is an unaffected population of subjects.

30. The method of claim 19, further comprising the step of performing additional diagnostic testing on a subject that has been identified as affected with Pompe disease.

31. A method of screening a neonatal subject for Pompe disease (glycogen storage disease type II), comprising the steps of determining the concentration of hexose tetrasaccharide (Glc)₄ in a biological sample taken from the neonatal subject, wherein (Glc)₄ has the structure α-D-Glc(1→6)-α-D-Glc(1→4)-α-D-Glc(1→4)-D-Glc, and comparing the concentration to a reference value;
wherein the detection of (Glc)₄ in the biological sample at more than the reference value identifies the neonatal subject as affected with Pompe Disease.

32. A method of monitoring the clinical condition of a subject with Pompe disease (glycogen storage disease II), comprising the steps of: determining the concentration of hexose tetrasaccharide (Glc)₄ in a biological sample taken from the subject, and comparing the concentration to a reference value;
wherein the detection of (Glc)₄ in the biological sample at more than the reference value is indicative of the clinical condition of the subject.

33. The method of claim 32, wherein (Glc)₄ has the structure α-D-Glc(1→6)-α-D-Glc(1→4)-α-D-Glc(1→4)-D-Glc.

34. The method of claim 32, wherein the subject is undergoing treatment for Pompe disease.

35. The method of claim 34, wherein the treatment is selected from the group consisting of enzyme replacement therapy, gene therapy, or dietary therapy.

36. The method of claim 34, wherein said monitoring is carried out to determine whether to commence or re-initiate treatment of the subject for Pompe disease.

37. A method of assessing the efficacy of a therapeutic regime in a subject with Pompe disease (glycogen storage disease type II), comprising the steps of: determining the concentration of hexose tetrasaccharide (Glc)₄ in a biological sample taken from the subject, and comparing the concentration to a reference value;
wherein the detection of (Glc)₄ in the biological sample at more than the reference value is indicative of the efficacy of the therapeutic regime in the subject.

38. The method of claim 37, wherein (Glc)₄ has the structure α-D-Glc(1→6)-α-D-Glc(1→4)-α-D-Glc(1→4)-D-Glc.

39. A method of screening a neonatal subject for Pompe disease (glycogen storage disease type II), comprising the steps of: determining the concentration of hexose tetrasaccharide (Glc)₄ by tandem mass spectrometry in a dried blood spot from the neonatal subject, and comparing the concentration to a reference value;
wherein the detection of (Glc)₄ in the biological sample at more than the reference value identifies the neonatal subject as affected with Pompe Disease.

40. The method of claim 39, wherein (Glc)₄ has the structure α-D-Glc(1→6)-α-D-Glc(1→4)-α-D-Glc(1→4)-D-Glc.

41. The method of claim 39, wherein the quantification by tandem mass spectrometry is standardized using a [U-¹³C]glucose labeled hexose tetramer as an internal standard.

42. The method of claim 41, wherein the internal standard comprises a [U-¹³C]glucose labeled hexose tetramer having the structure α-D-Glc(1→6)-α-D-Glc(1→4)-α-D-Glc(1→4)-D-Glc.

43. A method of determining the concentration of an oligosaccharide in a biological sample, comprising determining the concentration of hexose tetrasaccharide (Glc)₄ by tandem mass spectrometry in a biological sample taken from a subject.

44. The method of claim 43, wherein (Glc)₄ has the structure α-D-Glc(1→6)-α-D-Glc(1→4)-α-D-Glc(1→4)-D-Glc.

45. The method of claim 43, wherein the oligosaccharides in the biological sample are derivatized with butyl para-aminobenzoic acid prior to quantification by tandem mass spectrometry.

46. The method of claim 43, wherein the method further comprises a concentration step prior to said quantifying step.

47. The method of claim 46, wherein said concentration step comprises immunoprecipitation.

48. The method of claim 43, wherein the biological sample is, selected from the group consisting of blood, plasma, serum, urine, sputum, and amniotic fluid.

49. The method of claim 43, wherein the biological sample is selected from the group consisting of blood, plasma, and serum.

50. The method of claim 43, wherein the biological sample is a neonatal blood sample.

51. The method of claim 43, wherein the biological sample is a neonatal urine sample.

52. The method of claim 43, further comprising the step of reducing the concentration of glucose in the biological sample prior to said quantifying step.

53. The method of claim 43, wherein the quantification by tandem mass spectrometry is standardized using a [U-¹³C]glucose labeled hexose tetramer as an internal standard.

54. The method of claim 52, wherein the internal standard comprises a [U-¹³C] labeled hexose tetramer having the structure α-D-Glc(1→6)-α-D-Glc(1→4)-α-D-Glc(1→4)-D-Glc.
